SEQUENCE LISTING (50 sequences)

SEQ ID NO: 1 - RNA, 47 nt, synthetic construct
  source 1..47: mol_type = other RNA
gggaaataagagagaaaagaagagtaagaagaaatataagagccacc

SEQ ID NO: 2 - RNA, 57 nt, synthetic construct
  source 1..57: mol_type = other RNA
gggaaataagagagaaaagaagagtaagaagaaatataagaccccggcgccgccacc

SEQ ID NO: 3 - RNA, 119 nt, synthetic construct
  source 1..119: mol_type = other RNA
tgataataggctggagcctcggtggccatgcttcttgccccttgggcctccccccagcccctcctccccttcctgcacccgtacccccgtggtctttgaataaagtctgagtgggcggc

SEQ ID NO: 4 - RNA, 119 nt, synthetic construct
  source 1..119: mol_type = other RNA
tgataataggctggagcctcggtggcctagcttcttgccccttgggcctccccccagcccctcctccccttcctgcacccgtacccccgtggtctttgaataaagtctgagtgggcggc

SEQ ID NO: 5 - RNA, 75 nt, synthetic construct
  source 1..75: mol_type = other RNA
ggaaatcgcaaaatttgctcttcgcgttagatttcttttagttttctcgcaactagcaagctttttgttctcgcc

SEQ ID NO: 6 - RNA, 203 nt, synthetic construct
  source 1..203: mol_type = other RNA
ggaaatccccacaaccgcctcatatccaggctcaagaatagagctcagtgttttgttgtttaatcattccgacgtgttttgcgatattcgcgcaaagcagccagtcgcgcgcttgcttttaagtagagttgtttttccacccgtttgccaggcatctttaatttaacatatttttatttttcaggctaacctacgccgccacc

SEQ ID NO: 7 - RNA, 76 nt, synthetic construct
  source 1..76: mol_type = other RNA
ggaaataagagagaaaagaagagtaagaagaaatataagatctccctgagcttcagggagccccggcgccgccacc

SEQ ID NO: 8 - RNA, 89 nt, synthetic construct
  source 1..89: mol_type = other RNA
ggaaaccccccacccccgtaagagagaaaagaagagtaagaagaaatataagatctccctgagcttcagggagccccggcgccgccacc

SEQ ID NO: 9 - RNA, 59 nt, synthetic construct
  source 1..59: mol_type = other RNA
ggagaacttccgcttccgttggcgcaagcgctttcattttttctgctaccgtgactaag

SEQ ID NO: 10 - RNA, 46 nt, synthetic construct
  source 1..46: mol_type = other RNA
ggaaataagagagaaaagaagagtaagaagaaatataagagccacc

SEQ ID NO: 11 - RNA, 56 nt, synthetic construct
  source 1..56: mol_type = other RNA
ggaaataagagagaaaagaagagtaagaagaaatataagaccccggcgccgccacc

SEQ ID NO: 12 - RNA, 81 nt, synthetic construct
  source 1..81: mol_type = other RNA
ggaaatcgcaaaatttgctcttcgcgttagatttcttttagttttctcgcaactagcaagctttttgttctcgccgccgcc

SEQ ID NO: 13 - RNA, 81 nt, synthetic construct
  source 1..81: mol_type = other RNA
ggaaatcgcaaaattttcttttcgcgttagatttcttttagttttctttcaactagcaagctttttgttctcgccgccgcc

SEQ ID NO: 14 - RNA, 83 nt, synthetic construct
  source 1..83: mol_type = other RNA
  misc_difference 14..18: note = may be absent
  misc_difference 19: note = may be absent
  misc_difference 22: note = may be absent
  misc_difference 23..27: note = may be absent
  misc_difference 53: note = may be absent
  misc_difference 54: note = may be absent
  misc_difference 81..83: note = 81..83
ggaaatcgcaaaatttttgctctttttcgcgttagatttcttttagttttctykcaactagcaagctttttgttctcgccrcc

SEQ ID NO: 15 - RNA, 79 nt, synthetic construct
  source 1..79: mol_type = other RNA
ggaaaattttagcctggaacgttagataactgtcctgttgtctttatatacttggtccccaagtagtttgtcttccaaa

SEQ ID NO: 16 - RNA, 79 nt, synthetic construct
  source 1..79: mol_type = other RNA
ggaaactttatttagtgttactttattttctgtttatttgtgtttcttcagtgggtttgttctaatttccttggccgcc

SEQ ID NO: 17 - RNA, 79 nt, synthetic construct
  source 1..79: mol_type = other RNA
ggaaaatctgtattaggttggcgtgttctttggtcggttgttagtattgttgttgattcgtttgtggtcggttgccgcc

SEQ ID NO: 18 - RNA, 79 nt, synthetic construct
  source 1..79: mol_type = other RNA
ggaaaattattaacatcttggtattctcgataaccattcgttggattttattgtattcgtagtttgggttcctgccgcc

SEQ ID NO: 19 - RNA, 79 nt, synthetic construct
  source 1..79: mol_type = other RNA
ggaaattattattatttctagctacaatttatcattgtattattttagctattcatcattatttacttggtgatcaaca

SEQ ID NO: 20 - RNA, 79 nt, synthetic construct
  source 1..79: mol_type = other RNA
ggaaataggttgttaaccaagttcaagcctaataagcttggattctggtgacttgcttcaccgttggcgggcaccgatc

SEQ ID NO: 21 - RNA, 74 nt, synthetic construct
  source 1..74: mol_type = other RNA
ggaaatcgtagagagtcgtacttagtacatatcgactatcggtggacaccatcaagattataaaccaggccaga

SEQ ID NO: 22 - RNA, 79 nt, synthetic construct
  source 1..79: mol_type = other RNA
ggaaacccgcccaagcgaccccaacatatcagcagttgcccaatcccaactcccaacacaatccccaagcaacgccgcc

SEQ ID NO: 23 - RNA, 79 nt, synthetic construct
  source 1..79: mol_type = other RNA
ggaaagcgattgaaggcgtcttttcaactactcgattaaggttgggtatcgtcgtgggacttggaaatttgttgtttcc

SEQ ID NO: 24 - RNA, 79 nt, synthetic construct
  source 1..79: mol_type = other RNA
ggaaactaatcgaaataaaagagccccgtactcttttatttctattaggttaggagccttagcatttgtatcttaggta

SEQ ID NO: 25 - RNA, 79 nt, synthetic construct
  source 1..79: mol_type = other RNA
ggaaatgtgatttccagcaacttcttttgaatatattgaattcctaattcaaagcgaacaaatctacaagccatatacc

SEQ ID NO: 26 - RNA, 74 nt, synthetic construct
  source 1..74: mol_type = other RNA
ggaaatcgtagagagtcgtacttacgtggtcgccattgcatagcgcgcgaaagcaacaggaacaagaacgcgcc

SEQ ID NO: 27 - RNA, 74 nt, synthetic construct
  source 1..74: mol_type = other RNA
ggaaatcgtagagagtcgtacttagaataaacagagtcgggtcgacttgtctctgatactacgacgtcacaatc

SEQ ID NO: 28 - RNA, 79 nt, synthetic construct
  source 1..79: mol_type = other RNA
ggaaaatttgccttcggagttgcgtatcctgaactgcccagcctcctgatatacaactgttccgcttattcgggccgcc

SEQ ID NO: 29 - RNA, 79 nt, synthetic construct
  source 1..79: mol_type = other RNA
ggaaatctgagcaggaatcctttgtgcattgaagactttagattcctctctgcggtagacgtgcacttataagtatttg

SEQ ID NO: 30 - RNA, 79 nt, synthetic construct
  source 1..79: mol_type = other RNA
ggaaagcgattgaaggcgtcttttcaactactcgattaaggttgggtatcgtcgtgggacttggaaatttgttgccacc

SEQ ID NO: 31 - RNA, 104 nt, synthetic construct
  source 1..104: mol_type = other RNA
ggaaatttttttttgatattataagagtttttttttgatattaagaaaatttttttttgatattagaagagtaagaagaaatataagaccccggcgccgccacc

SEQ ID NO: 32 - RNA, 57 nt, synthetic construct
  source 1..57: mol_type = other RNA
ggaaataagagagaaaagaagagtaagaagaaatataagagccaaaaaaaaaaaacc

SEQ ID NO: 33 - RNA, 76 nt, synthetic construct
  source 1..76: mol_type = other RNA
ggaaatctccctgagcttcagggagtaagagagaaaagaagagtaagaagaaatataagaccccggcgccgccacc

SEQ ID NO: 34 - skipped

SEQ ID NO: 35 - RNA, 36 nt, synthetic construct
  source 1..36: mol_type = other RNA
ggactcactatttgttttcgcgcccagttgcaaaaa

SEQ ID NO: 36 - RNA, 119 nt, synthetic construct
  source 1..119: mol_type = other RNA
taaagctccccgggggcctcggtggcctagcttcttgccccttgggcctccccccagcccctcctccccttcctgcacccgtacccccgtggtctttgaataaagtctgagtgggcggc

SEQ ID NO: 37 - RNA, 119 nt, synthetic construct
  source 1..119: mol_type = other RNA
taagtctaagctggagcctcctgagagacctgtgtgaactattgagaagatcggaacagctccttactctgaggaagttggtacccccgtggtctttgaataaagtctgagtgggcggc

SEQ ID NO: 38 - RNA, 185 nt, synthetic construct
  source 1..185: mol_type = other RNA
taaagctccccggggcaaacaccattgtcacactccagcctcggtggcctagcttcttgccccttgggcccaaacaccattgtcacactccatccccccagcccctcctccccttcctgcacccgtaccccccaaacaccattgtcacactccagtggtctttgaataaagtctgagtgggcggc

SEQ ID NO: 39 - RNA, 170 nt, synthetic construct
  source 1..170: mol_type = other RNA
taaagctccccggggtccataaagtaggaaacactacagctggagcctcggtggcctagcttcttgccccttgggcccaaacaccattgtcacactccatccccccagcccctcctccccttcctgcacccgtacccccgtggtctttgaataaagtctgagtgggcggc

SEQ ID NO: 40 - RNA, 141 nt, synthetic construct
  source 1..141: mol_type = other RNA
taaagctccccgggggcctcggtggcctagcttcttgccccttgggcctccccccagcccctcctccccttcctgcacccgtaccccccaaacaccattgtcacactccagtggtctttgaataaagtctgagtgggcggc

SEQ ID NO: 41 - RNA, 185 nt, synthetic construct
  source 1..185: mol_type = other RNA
taagcccctccggggcaaacaccattgtcacactccagcctcggtggcctagcttcttgccccttgggcccaaacaccattgtcacactccatccccccagcccctcctccccttcctgcacccgtaccccccaaacaccattgtcacactccagtggtctttgaataaagtctgagtgggcggc

SEQ ID NO: 42 - RNA, 187 nt, synthetic construct
  source 1..187: mol_type = other RNA
taagcccctccggggtccataaagtaggaaacactacagcctcggtggcctagcttcttgccccttgggcctccataaagtaggaaacactacatccccccagcccctcctccccttcctgcacccgtaccccccgcattattactcacggtacgagtggtctttgaataaagtctgagtgggcggc

SEQ ID NO: 43 - RNA, 170 nt, synthetic construct
  source 1..170: mol_type = other RNA
taagcccctccggggtccataaagtaggaaacactacagctggagcctcggtggcctagcttcttgccccttgggcccaaacaccattgtcacactccatccccccagcccctcctccccttcctgcacccgtacccccgtggtctttgaataaagtctgagtgggcggc

SEQ ID NO: 44 - RNA, 141 nt, synthetic construct
  source 1..141: mol_type = other RNA
taagcccctccgggggcctcggtggcctagcttcttgccccttgggcctccccccagcccctcctccccttcctgcacccgtaccccccaaacaccattgtcacactccagtggtctttgaataaagtctgagtgggcggc

SEQ ID NO: 45 - protein, 883 aa, unidentified
  source 1..883: note = Escherichia phage T7
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 46 - protein, 884 aa, synthetic construct
MNTINIAKNDFSDIELAAIPFNTLADHYGERLAREQLALEHESYEMAEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFAG

SEQ ID NO: 47 - protein, 884 aa, synthetic construct
MNTINIAKNDFSDIELAAIPFNTLADHYGERLAREQLALEHESYEMAEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVWVIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFAG

SEQ ID NO: 48 - protein, 883 aa, synthetic construct
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVWVIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 49 - protein, 883 aa, synthetic construct; identical to SEQ ID NO: 48

SEQ ID NO: 50 - RNA, 10 nt, synthetic construct
  source 1..10: mol_type = other RNA
gcatatgact

(19) United States
(12) Patent Application Publication
(10) Pub. No.: US 2025/0369028 A1
(43) Pub. Date: Dec. 4, 2025

(54) CONTINUOUS IN VITRO TRANSCRIPTION PROCESS AND APPARATUS

(71) Applicant: ModernaTX, Inc., Cambridge, MA (US)

(72) Inventors: Matthew Burak, Cambridge, MA (US); Margaret Franklin, Cambridge, MA (US); Matthew Heinrich, Cambridge, MA (US); Tahir Kapoor, Cambridge, MA (US); Justin Riopel, Cambridge, MA (US); Yuxin He, Cambridge, MA (US)

(73) Assignee: ModernaTX, Inc., Cambridge, MA (US)

(21) Appl. No.: 19/210,716

(22) Filed: May 16, 2025

Related U.S. Application Data
(60) Provisional application No. 63/648,575, filed on May 16, 2024.

(51) Int. Cl. C12P 19/34; C12N 9/12; C12N 9/22; C12Q 1/6806

(52) U.S. Cl. CPC C12P 19/34; C12N 9/1247; C12N 9/22; C12Q 1/6806; C12Y 207/07006; C12Y 301/21001

(57) ABSTRACT

Some aspects of the disclosure relate to continuous in vitro transcription (IVT) methods in which Raman spectra are obtained to monitor the progress of an IVT reaction and adapt residence time accordingly. Also provided are continuous IVT reaction apparatuses for monitoring by Raman spectroscopy.

### RELATED APPLICATION

This application claims the benefit under 35 U.S.C. § 119 (e) of U.S. provisional application No. 63/648,575, filed May 16, 2024, the contents of which are incorporated by reference herein in their entirety.

### REFERENCE TO AN ELECTRONIC SEQUENCE LISTING

The instant application includes an electronic sequence listing (M137870294US01-SEQ-NTJ.xml; Size: 54,547 bytes; Date of Creation: May 16, 2025), the contents of which are incorporated by reference herein in their entirety.

### BACKGROUND

In vitro transcription (IVT) uses bacteriophage DNA-dependent ribonucleic acid (RNA) polymerases (e.g., SP6, T3 and T7 RNA polymerases) to synthesize DNA template-directed mRNA transcripts. IVT reactions are commonly “batch” reactions in that several reagents, including nucleoside triphosphates (NTPs), magnesium, RNA polymerase, deoxyribonucleic acid (DNA), and pyrophosphatase are combined at the beginning of the reaction. The components are then incubated, and the reaction proceeds until at least one of the NTPs is depleted. Thus, the reaction has at least one limiting reagent that may cause low yield of the RNA transcript (product). Other potential shortcomings of IVT reactions include, for example, abortive (truncated) transcripts, run-on transcripts, polyA tail variants producing 3′ heterogeneity, mutated transcripts, and/or double-stranded contaminants produced during the reactions.

### SUMMARY

Aspects of the disclosure relate to use of Raman spectroscopy to monitor and modify in vitro transcription (IVT) reaction conditions. It was discovered that the rate of IVT is sensitive to the nucleotide sequence to be transcribed (i.e., under a given set of reaction conditions, some DNA sequences will be transcribed faster or slower than others). Variability in IVT transcription rates (reaction rates) can reduce the efficiency of IVT, as the availability of NTPs and other reaction components may become rate-limiting in reactions progressing too quickly. Conversely, in a continuous process, reactions progressing too slowly can lead to output of IVT reaction mixtures containing excess amounts of NTPs that are discarded during RNA purification, leading to wasted reagents and reduced efficiency (in RNA produced per unit time and reactant cost). It was unexpectedly discovered that Raman spectroscopy allows monitoring of IVT reaction mixture kinetics (e.g., rate of NTP consumption), and that observed reaction rates indicate whether an IVT reaction mixture is proceeding slower or faster than a rate that is optimal for the RNA sequence being transcribed. Observed reaction kinetics were transformed to target residence times, which provide the most efficient reaction rate for transcribing that RNA sequence in a continuous process, achieving optimal RNA productivity and purity and minimal waste of NTPs and other reactants.

Methods of continuous IVT using Raman spectroscopy may be implemented using multiple types of reaction apparatus and processes for monitoring reactions. As one example, a continuous reactor (e.g., continuous stir tank reactor (CSTR)), receiving input solution(s) to form an IVT reaction mixture and outputting that IVT reaction mixture may be monitored, and the input and output flow rates adjusted to maintain a target residence time. As another example, a plug flow reactor (PFR), through which an IVT reaction mixture is flowing, may be monitored by Raman spectroscopy at one or more points along the flow path to determine the reaction rate, determine a target residence time, and the flow may be adjusted to maintain that target residence time. As another example, an output end of a PFR may be monitored to determine whether the IVT reaction does not reach an endpoint by the time the IVT reaction mixture reaches the end. As another example, a preliminary IVT reaction may be carried out and monitored to determine a target residence time for the sequence to be transcribed, and the flow length (residence time) of an IVT reaction in a PFR may be set to achieve that target residence time. Some aspects relate to continuous reaction apparatuses for continuous IVT, in which Raman spectrometers may be positioned to monitor IVT reaction rates.

Accordingly, some aspects relate to an in vitro transcription (IVT) method, the method comprising: (i) in a continuous reaction apparatus, incubating an IVT reaction mixture comprising a buffer, magnesium, a DNA, an RNA polymerase, a cap analog, adenosine triphosphate (ATP), cytidine triphosphate (CTP), guanosine triphosphate (GTP), and uridine triphosphate (UTP), whereby the RNA polymerase transcribes the DNA to produce an mRNA; wherein the IVT reaction mixture is formed by adding a first feed solution to the continuous reaction apparatus at a first input feed rate and a second feed solution to the continuous reaction apparatus at a second input feed rate, wherein the IVT reaction mixture is output from the continuous reaction apparatus at a first output flow rate; (iii) obtaining Raman spectra from the IVT reaction mixture over time; (iv) determining a reaction rate and a target endpoint from the Raman spectra; (v) determining a target residence time from the reaction rate and target endpoint; (vi) modifying the first input feed rate, second input feed rate, and/or first output flow rate such that a residence time of the IVT reaction mixture in the continuous reaction apparatus is 80% to 120% of the target residence time.

In some embodiments, the target residence time is determined by calculating a target reaction volume from the Raman spectra. In some embodiments, the residence time of the IVT reaction mixture is modified by modifying a total input feed rate, the total input feed rate being the sum of the first input feed rate and the second input feed rate. In some embodiments, the residence time of the IVT reaction mixture is modified by modifying the first output flow rate.

Some aspects relate to an in vitro transcription method, the method comprising: (i)(a) in a preliminary reaction apparatus, incubating a preliminary in vitro transcription (IVT) reaction mixture comprising a buffer, magnesium, a DNA, an RNA polymerase, a cap analog, adenosine triphosphate (ATP), cytidine triphosphate (CTP), guanosine triphosphate (GTP), and uridine triphosphate (UTP), whereby the RNA polymerase transcribes the DNA to produce an mRNA; (i)(b) obtaining Raman spectra from the preliminary IVT reaction mixture over time; (i)(c) determining a reaction rate and a target endpoint from the Raman spectra; (i)(d) determining a target residence time from the reaction rate and target endpoint; and (ii) in a continuous reaction apparatus comprising a plug flow reactor (PFR), incubating an in vitro transcription (IVT) reaction mixture flowing through the PFR at 80% to 120% of the target residence time, the IVT reaction mixture comprising a buffer, magnesium, a DNA, an RNA polymerase, a cap analog, adenosine triphosphate (ATP), cytidine triphosphate (CTP), guanosine triphosphate (GTP), and uridine triphosphate (UTP), whereby the RNA polymerase transcribes the DNA to produce the mRNA, wherein the IVT reaction mixture is output from the continuous reaction apparatus at a first output flow rate.

Some aspects relate to in vitro transcription (IVT) method, the method comprising, in a continuous reaction apparatus comprising a plug flow reactor (PFR): (i) incubating an in vitro transcription (IVT) reaction mixture flowing through the PFR with a residence time, the IVT reaction mixture comprising a buffer, magnesium, a DNA, an RNA polymerase, a cap analog, adenosine triphosphate (ATP), cytidine triphosphate (CTP), guanosine triphosphate (GTP), and uridine triphosphate (UTP); (ii) obtaining Raman spectra from the IVT reaction mixture at two or more points along the PFR separated by a predetermined distance; (iii) determining a reaction rate and a target endpoint from the Raman spectra; (iv) determining a target residence time from the reaction rate and target endpoint; and (v) modifying the residence time such that the IVT reaction mixture flows through the PFR with 80% to 120% of the target residence time, wherein the IVT reaction mixture is output from the continuous reaction apparatus at a first output flow rate.

Some aspects relate to in vitro transcription method, the method comprising, in continuous reaction apparatus comprising a plug flow reactor (PFR), (i) incubating an IVT reaction mixture flowing through the PFR with a residence time; (ii) obtaining Raman spectra from the IVT reaction mixture at an outlet location of the PFR over time; (iii) determining a reaction rate and a target endpoint from the Raman spectra; (iv) determining a target residence time from the reaction rate and target endpoint; and (v) modifying the residence time such that the IVT reaction mixture flows through the PFR with 80% to 120% of the target residence time, wherein the IVT reaction mixture is output from the continuous reaction apparatus at a first output flow rate.

In some embodiments, an active length of the PFR is adjustable. In some embodiments, modifying the residence time comprises opening a valve upstream of a current outlet location, after determining that the target endpoint occurred prior to the IVT reaction mixture reaching an end of the active length of the PFR. In some embodiments, modifying the residence time comprises closing an outlet and opening a valve downstream of the outlet, after determining that the target endpoint did not occur prior to the IVT reaction mixture reaching an end of the active length of the PFR.

In some embodiments, an mRNA yield of at least 80% of a theoretical maximum mRNA yield occurs when the residence time of the IVT reaction mixture in the continuous reaction apparatus is the target residence time. In some embodiments, a reaction rate of at least 80% of a theoretical maximum reaction rate occurs when the residence time of the IVT reaction mixture in the continuous reaction apparatus is the target residence time. In some embodiments, a concentration of nucleotide triphosphates (NTPs) in the IVT reaction mixture being output at the first output flow rate is 20% or less of a concentration of NTPs input into the continuous reaction apparatus.

In some embodiments, the IVT reaction mixture output at the first output flow rate flows into an additional reaction apparatus, and wherein the method further comprises: (i) contacting the additional reaction apparatus with an additional feed solution comprising an additional buffer and a DNase to form a DNase reaction mixture; and (ii) incubating the DNase reaction mixture, whereby the DNase cleaves the DNA to produce one or more DNA fragments; and (iii) separating the mRNA from the one or more DNA fragments and one or more other impurities to obtain an isolated mRNA composition.

In some embodiments, the IVT reaction mixture flowing at the first output flow rate flows continuously into the additional reaction apparatus. In some embodiments, a DNase reaction mixture flows continuously from the additional reaction apparatus to an mRNA purification module. In some embodiments, the additional reaction apparatus is a continuous plug flow reactor (CPFR) having one or more curved pipes, wherein the DNase reaction mixture flows through the CPFR with a Dean number (De) of at least 30. In some embodiments, each of the one or more curved pipes comprises (a) a diameter, and (b) a curve having a radius that is 180% to 400% of the diameter. In some embodiments, the additional reaction apparatus has a pressure drop of 0.5 bar or less.

In some embodiments, separating the mRNA from the one or more DNA fragments and/or other impurities comprises: (a) tangential flow filtration; (b) oligo-dT chromatography; and/or (c) high performance liquid chromatography.

In some embodiments, the separating the mRNA from the one or more DNA fragments and/or other impurities comprises: (a) separating the mRNA from one or more DNA fragments by tangential flow filtration; followed by (b) separating the mRNA from one or more other impurities by oligo-dT chromatography.

In some embodiments, the isolated mRNA composition comprises 0.1% (wt/wt) or less of uncleaved DNA molecules.

In some embodiments, the RNA polymerase is a T7 RNA polymerase. In some embodiments, the T7 RNA polymerase comprises the amino acid sequence of any one of SEQ ID NOs: 45-49.

In some embodiments, wherein the UTP is N1-methylpseudouridine triphosphate. In some embodiments, the CTP is 5-methylcytidine triphosphate.

In some embodiments, an mRNA yield of the method is at least 0.1 grams per liter per hour (g·L⁻¹·hr⁻¹). In some embodiments, at least 80% of mRNAs produced have a polyadenosine (polyA) tail. In some embodiments, at least 80% of mRNAs produced have a predetermined expected size.

In some embodiments, the IVT reaction mixture is incubated for at least 8 hours.

Some aspects relate to apparatus comprising: (i) a first feed solution container; (ii) a second feed solution container; (iii)(a) a continuous in vitro transcription (IVT) reaction apparatus fluidically coupled downstream of both the first feed solution container and the second feed solution container which is configured to receive a first mixed inlet stream; and (iii)(b) a Raman sensor coupled to the continuous IVT reaction apparatus.

In some embodiments, the continuous IVT reaction apparatus is a plug flow reaction (PFR) comprising two or more Raman sensors configured to obtain Raman spectra from a solution flowing through the PFR at two or more points separated by a predetermined distance.

In some embodiments, the continuous IVT reaction apparatus is a plug flow reaction (PFR), wherein the Raman sensor is configured to obtain a Raman spectrum from an output end of the continuous IVT reaction apparatus.

In some embodiments, the apparatus further comprises one or more mRNA purification modules selected from the group consisting of: (a) a tangential flow filtration module; (b) an oligo-dT chromatography module; and/or (c) a high performance liquid chromatography (HPLC) module.

In some embodiments, the apparatus further comprises a DNase reaction apparatus (a) fluidically coupled downstream of the continuous IVT reaction apparatus, and (b) configured to receive a third feed solution comprising a DNase, the DNase reaction apparatus comprising a continuous plug flow reaction (CPFR) comprising one or more curved pipes. In some embodiments, each of the one or more curved pipes comprises (a) a diameter, and (b) a curve having a radius that is 180% to 400% of the diameter. In some embodiments, a solution flowing through the one or more curved pipes has a Dean number (De) of at least 30.

In some embodiments, the apparatus further comprises: (a) a tangential flow filtration module; and (b) an oligo-dT chromatography module, wherein the apparatus is configured to remove one or more DNA fragments from a mixture comprising an mRNA and the one or more DNA fragments, before the mRNA is introduced into the oligo-dT chromatography module.

These and other embodiments of continuous IVT methods and apparatuses are described in more detail in the Detailed Description.

### BRIEF DESCRIPTION OF THE DRAWINGS

FIG. 1A shows Raman spectra (plotting normalized intensity (AU) versus Raman shift (cm⁻¹)) of in vitro transcription of mRNA with gray-scale to show time (with black being the first spectrum and white being the last spectrum). Box A represents peak(s) associated with glycerol, box B represents peak(s) associated with orthophosphate (which is representative of total mRNA), box C represents peak(s) associated with total nucleoside triphosphates (NTPs), and box D represents peak(s) associated with individual NTPs.

FIG. 1B shows the normalized intensity (y-axis labeled Response 990) of the orthophosphate peak(s) (box B from FIG. 1A) versus time (in minutes) for the five batches from FIG. 1A. Box D represents a first timepoint, box E represents a second timepoint, and box F represents the endpoint.

FIG. 1C plots the Scores on PC1 versus time (in minutes) for the five batches from FIGS. 1A and 1B based on full spectral information using PCA.

FIG. 1D plots total mRNA (mg/mL) predicted by monitoring peak(s) associated with orthophosphate versus total mRNA (mg/mL) measured with HPLC.

FIG. 1E plots normalized intensity of a Raman peak at 730 cm⁻¹—representative of ATP—versus time for five batches of in vitro mRNA transcription.

FIG. 1F plots normalized intensity of a Raman peak at 1115 cm⁻¹—representative of total NTPs—versus time for five batches of in vitro mRNA transcription.

FIG. 1G plots normalized intensity of a Raman peak at 1576 cm⁻¹—representative of GTP—versus time for five batches of in vitro mRNA transcription.

FIG. 2A shows different rate constants for 10 different RNA sequences, which may be grouped as “slow”, “medium”, or “fast”, with each group including sequences of different lengths.

FIG. 2B shows modeling of rNTP concentrations (and indirectly reaction rates) as functions of sequence IVT rate constants and residence time, with desired reaction rates being achieved using variable residence times for sequences with different reaction rates.

FIGS. 2C and 2D show decreasing purity, in terms of polyA tailing efficiency (FIG. 2C) and expected size (FIG. 2D) with increasing residence time, indicating that shorter residence times increase mRNA purity.

FIG. 3A shows the correlation between predicted RNA concentration by Raman spectroscopy monitoring and measured RNA concentration.

FIG. 3B and FIG. 3C show kinetics of tailed mRNA concentrations over time, with overlaid predictions from Raman spectroscopy monitoring.

FIG. 4A shows IVT endpoint determination using the rate-based window method. DiMaso et al., React Chem Eng. 2020. 5:1642-1646.

FIG. 4B shows comparison of Raman-determined endpoints (right columns) with empirically measured endpoints using HPLC.

FIG. 5A shows a reaction control strategy in which input feed rates of IVT reaction mixture components are adjusted based on monitoring of reaction conditions in a CSTR. FIG. 5B shows RNA yield over time. FIG. 5C shows RNA tail purity over time. FIG. 5D shows RNA size purity over time.

FIGS. 5E-5G show rate constants (FIG. 5D), tail purity (FIG. 5E), and size purity (FIG. 5F) achieved by an IVT master mix following storage at 18 or 5° C. for 28 days, indicating that RNA polymerase and other reaction components are stable for at least 28 days at 5° C., allowing continuous IVT to continue for extended durations.

FIG. 6A shows a design of a continuous plug flow reactor (CPFR) including Dean vortices, for use in DNase digestion of IVT reaction mixtures.

FIG. 6B shows variance in rATP detected over time for a mixture flowing at 0.04 m/s.

FIG. 6C shows the relationship between flow velocity (v) and HETP/I.D. HETP=Height Equivalent to a Theoretical Plate. I.D.=inlet diameter.

FIG. 6D shows HETP as a function of Dean number (De), indicating that HETP/I.D. is consistent at De of at least 30.

FIG. 6E shows pDNA (% wt/wt) in output DNase digestion mixture over time. Black line indicates a target maximum DNA content of 0.1% wt/wt.

FIG. 7 shows an example of an apparatus and workflow for continuous IVT and continuous DNase digestion, with a TFF module for DNA fragment removal and an oligo-dT module for purification of mRNA.

### DETAILED DESCRIPTION

Provided herein are continuous IVT methods and reaction apparatuses for continuous IVT. It was surprisingly discovered that for a given set of reaction conditions (e.g., temperature, volume, reactant concentrations) rates of in vitro transcription are sensitive to the RNA sequence to be transcribed (i.e., RNA transcription progresses at different rates for different RNA sequences). Such sequence sensitivity poses a challenge for continuous IVT methods, as a fixed sequence-agnostic residence time can cause reduced productivity (in mass of mRNA per length of time) for sequences are transcribed more quickly, and increased costs (in excess reactants present in output material and lost during purification) for sequences that are transcribed more slowly. Additionally, excess residence time may allow aberrant double-stranded RNA production via RNA-templated transcription by RNA polymerases. As dsRNA stimulates innate immune responses in cells that can cause mRNA degradation, the presence of such dsRNA contaminants reduces the potency of mRNA compositions for therapeutic or prophylactic use, so reducing residence time to the extent possible provides additional benefits to the purity and potency of mRNA compositions.

### Continuous In Vitro Transcription (IVT) Methods and Apparatuses

Some aspects relate to methods of continuous in vitro transcription (IVT) in which Raman spectroscopy is used to monitor progress of the IVT reaction and adjust reaction conditions accordingly. In some embodiments, residence time of the IVT reaction mixture in a reaction apparatus is maintained at at least 70%, at least 75%, at least 80%, at least 85%, at least 90%, at least 91%, at least 92%, at least 93%, at least 94%, at least 95%, at least 96%, at least 97%, at least 98%, or at least 99% of the target residence time. In some embodiments, residence time of the IVT reaction mixture in a reaction apparatus is maintained at 130% or less, 125% or less, 120% or less, 115% or less, 110% or less, 109% or less, 108% or less, 107% or less, 106% or less, 105% or less, 104% or less, 103% or less, 102% or less, or 101% or less of the target residence time. In some embodiments, residence time of the IVT reaction mixture in a reaction apparatus is maintained at 70% to 130%, 75% to 125%, 80% to 120%, 85% to 115%, 90% to 110%, or 95% to 105% of the target residence time.

The skilled artisan will appreciate that how residence time is modified will depend on the specific reaction apparatus being employed. For example, in a continuous stir tank reactor (CSTR), the rates at which reactants are input and output may be modified to increase or decrease the amount of time a given unit of IVT reaction mixture (e.g., a given NTP molecule) spends in the reaction apparatus before being output. As another example, in a plug flow reactor (PFR), the length of the PFR may be modified, such that a given unit of IVT reaction mixture (e.g., a given NTP molecule) spends a given amount of time in the PFR after being input and before reaching an output end. As another example, the flow rate of IVT reaction mixture through a PFR may be modified to adjust the residence time.

In some aspects, a continuous IVT method comprises:
(i) in a continuous reaction apparatus, incubating an IVT reaction mixture comprising a buffer, magnesium, a DNA, an RNA polymerase, a cap analog, adenosine triphosphate (ATP), cytidine triphosphate (CTP), guanosine triphosphate (GTP), and uridine triphosphate (UTP), whereby the RNA polymerase transcribes the DNA to produce an mRNA;
wherein the IVT reaction mixture is formed by adding a first feed solution to the continuous reaction apparatus at a first input feed rate and a second feed solution to the continuous reaction apparatus at a second input feed rate,
wherein the IVT reaction mixture is output from the continuous reaction apparatus at a first output flow rate;
(iii) obtaining Raman spectra from the IVT reaction mixture over time;
(iv) determining a reaction rate and a target endpoint from the Raman spectra;
(v) determining a target residence time from the reaction rate and target endpoint;
(vi) modifying the first input feed rate, second input feed rate, and/or first output flow rate such that a residence time of the IVT reaction mixture in the continuous reaction apparatus is 80% to 120% of the target residence time.

In some embodiments, modification of residence time of the IVT reaction mixture is accomplished by modifying the first and/or second input feed rates. In some embodiments, modification of residence time of the IVT reaction mixture is accomplished by modifying the output flow rate. In some embodiments, the first and/or second input feed rate(s) and the output flow rate are modified such that the IVT reaction mixture having a given residence time in the apparatus has a consistent volume. In some embodiments, modifying the residence time of the IVT reaction mixture comprises reducing the volume of IVT reaction mixture present in the continuous reaction apparatus. In some embodiments, modifying the residence time of the IVT reaction mixture comprises increasing the volume of IVT reaction mixture present in the continuous reaction apparatus. In some embodiments, the volume of IVT reaction mixture present in the continuous reaction apparatus is maintained at 80% or less, 85% or less, 90% or less, or 95% or less the capacity of the continuous reaction apparatus.

In some embodiments, target residence time is determined by calculating a target reaction volume from the Raman spectra. In some embodiments, the volume of the IVT reaction mixture in a reaction apparatus is maintained at 130% or less, 125% or less, 120% or less, 115% or less, 110% or less, 109% or less, 108% or less, 107% or less, 106% or less, 105% or less, 104% or less, 103% or less, 102% or less, or 101% or less of the target volume. In some embodiments, the volume of the IVT reaction mixture in a reaction apparatus is maintained at 70% to 130%, 75% to 125%, 80% to 120%, 85% to 115%, 90% to 110%, or 95% to 105% of the target volume. In some embodiments, the volume of the IVT reaction mixture in a reaction apparatus is maintained at at least 70%, at least 75%, at least 80%, at least 85%, at least 90%, at least 91%, at least 92%, at least 93%, at least 94%, at least 95%, at least 96%, at least 97%, at least 98%, or at least 99% of the target volume.

Where a first and second feed solution are combined to form an IVT reaction mixture, first feed solutions comprise a buffer, NTPs, and a cap or cap analog; and second feed solutions comprise a buffer, RNA polymerase, and DNA. NTPs and caps (or cap analogs) are consumed as they are incorporated into RNA transcripts. By contrast, DNAs encoding an RNA transcript serve as templates for transcription, and RNA polymerases transcribe RNA from DNA templates, but neither the DNA nor RNA polymerase is consumed during IVT (i.e., they may be reused to produce multiple RNA transcripts). Thus, the first feed solution (comprising consumable NTPs and caps or cap analogs) may be input at a different feed rate than the second feed solution (comprising reusable DNA and RNA polymerase), to modify the input rate of consumable reagents while maintaining a given input rate of reusable components.

First and second feed solution input feed rates may be maintained at similar rates. While DNA and RNA polymerases may be reused in transcription, output of a reaction mixture from an apparatus removes DNA and RNA polymerase from the apparatus, and so both feed solutions may be input at similar rates to maintain a given balance of inputs and outputs. In some embodiments, the first and second feed solutions are input at substantially identical feed rates. In some embodiments, when one feed solution input rate is modified, the other feed solution input rate is modified in a similar manner, such that both the input feed rates remain substantially identical.

In some embodiments, the output flow rate is substantially identical to the first input feed rate and/or the second input feed rate. In some embodiments, when input feed rate(s) are modified, the output flow rate is modified in a similar manner, such that both the input feed rate(s) and output flow rate remain substantially identical.

First and second feed solutions may comprise the same buffer, or a different buffer. Any buffer suitable for IVT may be used. Non-limiting examples of buffers useful in IVT are described in International Application No. PCT/US2020/021955, which is incorporated by reference herein for this purpose.

In some embodiments, the first feed solution comprises magnesium. In some embodiments, the second feed solution comprises magnesium. In some embodiments, both the first feed solution and the second feed solution comprise magnesium. Magnesium is used as a cofactor by certain RNA polymerases.

An IVT reaction mixture may be continuously mixed in a continuous reaction apparatus. Continuous mixing maintains distribution of components in a mixture, thereby improving reaction efficiency by increasing the frequency of contact between components (e.g., RNA polymerase and DNA, RNA polymerase and NTPs). Mixing may be accomplished by any suitable method for the reactor being employed.

Any suitable reactor may be used as a continuous reaction apparatus. In some embodiments, the continuous reaction apparatus is a continuous stir tank reactor (CSTR).

In some aspects, a continuous IVT method comprises:
(i)(a) in a preliminary reaction apparatus, incubating a preliminary in vitro transcription (IVT) reaction mixture comprising a buffer, magnesium, a DNA, an RNA polymerase, a cap analog, adenosine triphosphate (ATP), cytidine triphosphate (CTP), guanosine triphosphate (GTP), and uridine triphosphate (UTP), whereby the RNA polymerase transcribes the DNA to produce an mRNA;
(i)(b) obtaining Raman spectra from the preliminary IVT reaction mixture over time;
(i)(c) determining a reaction rate and a target endpoint from the Raman spectra;
(i)(d) determining a target residence time from the reaction rate and target endpoint; and
(ii) in a continuous reaction apparatus comprising a plug flow reactor (PFR), incubating an in vitro transcription (IVT) reaction mixture flowing through the PFR at 80% to 120% of the target residence time, the IVT reaction mixture comprising a buffer, magnesium, a DNA, an RNA polymerase, a cap analog, adenosine triphosphate (ATP), cytidine triphosphate (CTP), guanosine triphosphate (GTP), and uridine triphosphate (UTP), whereby the RNA polymerase transcribes the DNA to produce the mRNA,
wherein the IVT reaction mixture is output from the continuous reaction apparatus at a first output flow rate.

Any suitable reactor may be used for a preliminary IVT reaction. The preliminary reaction apparatus need not be a continuous reaction apparatus. For example, in some embodiments, the preliminary reaction apparatus is a fed batch reactor. In some embodiments, the preliminary IVT reaction mixture is incubated for at least 1 hour, at least 2 hours, at least 3 hours, or at least 4 hours without the addition of a feed solution, while Raman spectra are being collected.

In some embodiments, the preliminary reaction apparatus is a CSTR. In some embodiments, first and second feed solutions as described in the preceding subsection are added to the CSTR, and the preliminary IVT reaction mixture is output at an output flow rate, while Raman spectra are being collected.

In some embodiments, a preliminary IVT reaction mixture is formed by input of one or more feed solutions into the preliminary reaction apparatus, and the same feed solution(s) are input into the PFR, such that an IVT reaction mixture at the beginning of the PFR active length has the same or substantially similar reactant concentrations. In some embodiments, formation of an IVT reaction mixture in the preliminary reaction apparatus is independent of input(s) into the PFR.

In some aspects, a continuous IVT method comprises, in a continuous reaction apparatus comprising a plug flow reactor (PFR):
(i) incubating an in vitro transcription (IVT) reaction mixture flowing through the PFR with a residence time, the IVT reaction mixture comprising a buffer, magnesium, a DNA, an RNA polymerase, a cap analog, adenosine triphosphate (ATP), cytidine triphosphate (CTP), guanosine triphosphate (GTP), and uridine triphosphate (UTP);
(ii) obtaining Raman spectra from the IVT reaction mixture at two or more points along the PFR separated by a predetermined distance;
(iii) determining a reaction rate and a target endpoint from the Raman spectra;
(iv) determining a target residence time from the reaction rate and target endpoint; and
(v) modifying the residence time such that the IVT reaction mixture flows through the PFR with 80% to 120% of the target residence time,
wherein the IVT reaction mixture is output from the continuous reaction apparatus at a first output flow rate.

In some embodiments, Raman spectra are obtained at 2, 3, 4, 5, 6, 7, 8, 9, 10, 11, 12, 13, 14, 15, 16, 17, 18, 19, or 20 points along the length of the PFR. In some embodiments, Raman spectra are obtained at 2-5, 5-10, 10-15, or 15-20 points along the length of the PFR. In some embodiments, Raman spectra are obtained at up to 5, up to 10, up to 15, or up to 20 points along the length of the PFR.

In some embodiments, the points at which two or more pairs of Raman spectra are obtained are separated by substantially equal distances. The skilled artisan will appreciate that evaluation of the distance between collection points considers individual pairs of points at which two Raman spectra are collected, where no additional Raman spectra are collected between those two points. For example, a series of three equally spaced collection points would have two pairs of collection points—a first and second, and the second and a third. Even though the first and third collection points are separated by a (2-fold) larger distance, a substantially equal distance separates each individual pair of collection points that do not include another collection point between them.

In some embodiments, the distance between a first and second point of Raman spectra collection differs from the distance between the second point and a third point of Raman spectrum collection. In some embodiments, the distance between the second and third collection points is shorter than the distance between the first and second collection points. As the reaction IVT mixture flows through a PFR and the IVT reaction progresses, shorter separation between Raman spectra collection points increases the resolution of reaction rates as the IVT reaction mixture approaches an endpoint.

In some aspects, a continuous IVT method comprises, in continuous reaction apparatus comprising a plug flow reactor (PFR),
(i) incubating an IVT reaction mixture flowing through the PFR with a residence time;
(ii) obtaining Raman spectra from the IVT reaction mixture at an outlet location of the PFR over time;
(iii) determining a reaction rate and a target endpoint from the Raman spectra;
(iv) determining a target residence time from the reaction rate and target endpoint; and
(v) modifying the residence time such that the IVT reaction mixture flows through the PFR with 80% to 120% of the target residence time,
wherein the IVT reaction mixture is output from the continuous reaction apparatus at a first output flow rate.

In some embodiments, modifying residence time comprises adjusting the position of input(s) into the PFR. For example, following a determination that residence time must be reduced to achieve the target residence time, IVT reaction mixture (or feed solutions that are combined in the PFR to form the IVT reaction mixture) may be input into the PFR at a position closer to the outlet location. Conversely, following a determination that residence time must be increased to achieve the target residence time, IVT reaction mixture (or feed solutions) may be input at a position farther away from the outlet location.

In some embodiments, modifying residence time comprises increasing the flow rate of IVT reaction mixture in the PFR. For example, flow rate may be increased if a shorter residence time is needed to achieve the target residence time, or decreased if a longer residence time is warranted.

In some embodiments, at least 70%, at least 75%, at least 80%, at least 85%, at least 90%, or at least 95% of mRNAs produced by a continuous IVT method comprise a poly(A) tail. In some embodiments, at least at least 70%, at least 75%, at least 80%, at least 85%, at least 90%, or at least 95% of mRNAs have a predetermined expected size. The skilled artisan will appreciate that expected size of an mRNA to be transcribed is determined by the starting and ending points of transcription from the DNA template, which may vary based on the sequence in question. In some embodiments, a continuous IVT method produces an mRNA yield of at least 0.1, at least 0.2, at least 0.3, at least 0.4, at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5 grams of mRNA per liter of IVT reaction mixture per hour (g·L⁻¹·hr⁻¹).

In some embodiments, a continuous IVT method comprises incubating an IVT reaction mixture for at least 8 hours, at least 12 hours, at least 18 hours, or at least 24 hours. In some embodiments, the method comprises incubating the IVT reaction mixture for at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 days. In some embodiments, the IVT reaction mixture is incubated for up to 20, up to 18, up to 16, up to 14, up to 12, or up to 10 days. Because continuous IVT methods involve continuous input into a system to form an IVT reaction mixture in the system and removal of some portion of the IVT reaction mixture from the system, the skilled artisan will appreciate that the duration for which IVT reaction mixture is incubated in a system is distinct from “residence time” as applied to a specific unit of IVT reaction mixture. Rather, the duration of incubating an IVT reaction mixture refers to the time for which the continuous IVT reaction mixture is run.

### Target Endpoint

Embodiments of continuous IVT methods include determining a target endpoint from Raman spectra obtained by monitoring an IVT reaction (e.g., the continuous IVT reaction, or a preliminary IVT reaction to inform residence time for the continuous IVT reaction). Multiple criteria may be used to determine an endpoint, such as a target amount of RNA transcript production, a target amount of NTPs consumption, or a target amount of cap or cap analog consumption. In some embodiments, the target endpoint is defined as the concentration of one or more NTPs being output from the continuous reaction apparatus is 30% or less, 25% or less, 20% or less, 15% or less, 10% or less, or 5% or less than the concentration of the one or more NTPs input into the continuous reaction apparatus. In some embodiments, the target endpoint is defined as the concentration of cap being output from the continuous reaction apparatus is 30% or less, 25% or less, 20% or less, 15% or less, 10% or less, or 5% or less than the concentration of the cap being input into the continuous reaction apparatus. In some embodiments, the target endpoint is defined as the concentration of cap analog being output from the continuous reaction apparatus is 30% or less, 25% or less, 20% or less, 15% or less, 10% or less, or 5% or less than the concentration of the cap analog being input into the continuous reaction apparatus. The relative concentrations of NTP(s) and/or caps or cap analogs being input into and output from the continuous reaction apparatus may be measured at any time, including when Raman spectra are being collected, immediately after modifying residence time, and/or the IVT reaction has reached a steady state after the most recent modification of residence time.

An endpoint may be defined using conversion. Conversion is calculated using the following equation.

$$Conversion = \frac{moles of reactant reacted}{moles of reactant fed} = \frac{{F}_{A 0} - {F}_{A}}{{F}_{A 0}} \qquad Eq . 1$$

Other metrics may be employed to quantify when the reaction has reached, or will reach, an endpoint. These include but are not limited to the reaction reaching a predetermined reaction rate, the first derivative of a reaction reaching a predetermined value, the reaction achieving a desired yield, or any other reaction metric which can be used to determine the progress of the reaction. A target endpoint may be established which selects a threshold value for a reaction progress metric which is compared to the actual reaction progress to identify whether the reaction progress metric is above or below the threshold.

### Residence Time

Embodiments of continuous IVT methods include determining a target residence time, based on the reaction rate and target endpoint determined from Raman spectra. Residence time is defined as the average time that a unit of feed solution stays in the continuous reaction apparatus before being output from the continuous reaction apparatus. Residence time may calculated using any suitable method for the reactor being used. In some instances, the residence time is a function of the input feed rate(s), output flow rate, and volume of the reaction apparatus. Specific applications of residence time calculations can be found below in their respective reactor descriptions. In general, residence time can be calculated using the following equation.

$$residence time = \frac{reactor volume}{volumetric flow rate} \qquad Eq . 2$$

Target residence time is defined as the residence time that allows an IVT reaction to reach the target endpoint. Accordingly, residence time may be modified, following calculation of reaction rate and target endpoint from Raman spectra, to be within a desired window of the target residence time (e.g., 80% to 120%). Additionally, using the target residence time and a target product flow rate, a desired reaction volume may be calculated using Equation 2.

In some embodiments, the target residence time is defined by an mRNA yield, when the IVT reaction is proceeding at the target residence time, that is at least 70%, at least 75%, at least 80%, at least 85%, at least 90%, or at least 95% of a theoretical maximum mRNA yield.

In some embodiments, the target residence time is defined by a reaction rate, when the IVT reaction is proceeding at the target residence time, that is at least 70%, at least 75%, at least 80%, at least 85%, at least 90%, or at least 95% of a theoretical maximum reaction rate. The mRNA yield and reaction rate may be measured at any time, including when Raman spectra are being collected, immediately after modifying residence time, and/or the IVT reaction has reached a steady state after the most recent modification of residence time.

### Raman Spectra

Continuous IVT methods include collection of Raman spectra from the IVT reaction mixture to monitor one or more conditions of the IVT reaction, such as mRNA transcription rate and/or the rate of consumption of one or more reactants (e.g., NTPs). Use of Raman spectroscopy to monitor IVT reactions is described, e.g., in International Application No. PCT/US2022/028969, which is incorporated by reference herein for this purpose.

In some embodiments, Raman spectra are obtained to monitor the formation of one or more byproducts (e.g., orthophosphate) over time. During IVT, incorporation of an NTP into an RNA transcript results in release of inorganic pyrophosphate (PPi), which may be hydrolyzed to produce orthophosphate, as shown in the following equations:

$${( RNA )}_{n} + {MgNTP ( RNA )}_{n + 1} \to {MgP}_{2} {O}_{7}^{2 -} + {H}^{+} \\ {MgP}_{2} {O}_{7}^{2 -} + {H}_{2} O ⇄ 2 {HPO}_{4}^{2 -} + {Mg}^{2 +}$$

Accordingly, in some embodiments, the rate of production of mRNA, the amount of mRNA produced, and/or the stage of the in vitro transcription (e.g., relative to endpoint) may be determined from the rate of formation of orthophosphate and/or the amount of orthophosphate produced.

In certain embodiments, a method comprises monitoring one or more peaks (e.g., monitoring the appearance of a peak and/or the increase in size of a peak) at about 970-1000 cm⁻¹. For example, in some embodiments, a method comprises monitoring the formation of orthophosphate by monitoring a peak (e.g., monitoring intensity of a peak) at about 970-1000 cm⁻¹.

In some embodiments, Raman spectra are obtained to monitor the reduction in concentration of one or more reactants, such as one or more NTPs (e.g., ATP, GTP, CTP, and/or UTP, and/or total NTPs) (e.g., over time) (e.g., of one or more Raman spectra). During in vitro transcription, each NTP addition results in a reduction in concentration of an NTP, such that the rate of production of mRNA, the amount of mRNA produced, and/or the stage of the in vitro transcription (e.g., relative to the endpoint) may be determined based on the rate of reduction of one or more NTPs (e.g., total NTPs) and/or the amount of one or more NTPs (e.g., total NTPS), in certain embodiments. In some embodiments, the concentration of one or more NTPs is monitored by monitoring a peak at about 700-800 cm⁻¹ and/or 1100-1700 cm⁻¹ (e.g., 1150-1650 cm⁻¹). For example, according to some embodiments, the concentration of GTP is monitored by monitoring a peak at about 1560-1600 cm⁻¹ (e.g., 1570-1590 cm⁻¹) and/or a peak at about 1470-1500 cm⁻¹ (e.g., 1480-1495 cm⁻¹). According to certain embodiments, the concentration of ATP is monitored by monitoring a peak at about 710-750 cm⁻¹ (e.g., 720-740 cm⁻¹). According to some embodiments, the concentration of CTP is monitored by monitoring a peak at about 770-800 cm⁻¹ (e.g., 775-790 cm⁻¹). According to some embodiments, the concentration of UTP is monitored by monitoring a peak at about 780-810 cm⁻¹ (e.g., 785-805 cm⁻¹), 1220-1240 cm⁻¹, and/or 1660-1680 cm⁻¹. In certain embodiments, the concentration of total NTPs is monitored by monitoring a peak at about 1100-1120 cm⁻¹. In some embodiments, the concentration of an individual NTP is monitored and/or determined by monitoring and/or determining the concentration of total NTPs and the concentration of the other individual NTPs present. For example, if GTP, ATP, CTP, and UTP were present, the concentration of UTP may be monitored and/or determined by monitoring and/or determining the total NTP concentration and the concentrations of GTP, ATP, and CTP, and subtracting those concentrations from the total NTP concentration.

In certain embodiments, a method comprises monitoring one or more peaks (e.g., monitoring the disappearance of a peak and/or the decrease in size of a peak) at about 1100-1120 cm⁻¹ and/or one at about 1150-1650 cm⁻¹. For example, in some embodiments, the method comprises monitoring the reduction in concentration of one or more NTPs by monitoring one or more peaks at about 1100-1120 cm⁻¹. As another example, in certain embodiments, the method comprises monitoring the reduction in concentration of total NTPs by monitoring one or more peaks at about 1150-1650 cm⁻¹.

In some embodiments, a method comprises monitoring concentration of one or more components of one or more enzyme solutions (e.g., of one or more Raman spectra) (e.g., over time). In some embodiments, the concentration of one or more components of one or more enzyme solutions is monitored by monitoring one or more peaks at about 800-880 cm⁻¹. For example, in certain embodiments, the concentration of glycerol in one or more enzyme solutions is monitored by monitoring one or more peaks at about 800-880 cm⁻¹.

In some embodiments, a method comprises monitoring concentration of one or more components of one or more buffers (e.g., of one or more Raman spectra) (e.g., over time). In some embodiments, the concentration of one or more components of a buffer is monitoring by monitoring one or more peaks at about 920-940 cm⁻¹ and/or one or more peaks at about 1040-1070 cm⁻¹. For example, in some embodiments, the concentration of acetate in one or more buffers is monitored by monitoring one or more peaks at about 920-940 cm⁻¹. In certain embodiments, the concentration of tris in one or more buffers is monitored by monitoring one or more peaks at about 1040-1070 cm⁻¹.

In some embodiments, the method (e.g., monitoring and/or the determining) comprises using an algorithm (e.g., to analyze the Raman spectra) (e.g., to determine whether the in vitro transcription has reached a desired endpoint, whether the in vitro transcription is progressing at a desired rate, and/or whether one or more reaction conditions are as desired). In certain embodiments, the algorithm comprises Principal Component Analysis (PCA) and/or a Batch Evolution Model. According to certain embodiments, PCA transforms the data for each variable (e.g., the data for each wavenumber) into data for a new set of variables called Principal Components (PCs). In certain embodiments, PC1 is a combination of wavenumbers with the largest change over time, while PC2 is a combination of wavenumbers with the second largest change over time, and so on. According to some embodiments, plotting PC1 over time generates a single curve representative of the overall changes in the spectra over time. In accordance with some embodiments, the single curve generated by PCA (e.g., for one batch) (e.g., PC1) may be compared to another single curve generated by PCA (e.g., for another batch and/or a desired PCA curve) (e.g., PC1) rather than comparing the spectra directly.

According to some embodiments, the method (e.g., monitoring and/or the determining) comprises comparing one or more Raman spectra and/or representations thereof (e.g., spectra analyzed by an algorithm, such as PCA and/or a Batch Evolution Model) to one or more reference Raman spectra and/or representations thereof (e.g., from a prior batch) to identify the presence of one or more differences. In certain embodiments, the presence of one or more differences may be identified through visual comparison and/or statistical analysis. In some embodiments, the method further comprises identifying the cause of the one or more differences that are present (e.g., differences in one or more reaction conditions).

### Reaction Apparatuses

Some aspects relate to continuous IVT methods using continuous reaction apparatuses. Some aspects relate to apparatuses themselves that are suitable for continuous IVT methods. Unless otherwise clear from context, the skilled artisan will appreciate that descriptions of apparatuses are equally relevant to apparatuses themselves and to continuous IVT methods using continuous reaction apparatuses.

In some aspects, an apparatus comprises:
(i) a first feed solution container;
(ii) a second feed solution container;
(iii)(a) a continuous in vitro transcription (IVT) reaction apparatus fluidically coupled downstream of both the first feed solution container and the second feed solution container which is configured to receive a first mixed inlet stream; and
(iii)(b) a Raman sensor coupled to the continuous IVT reaction apparatus.

In some embodiments, the continuous IVT reaction apparatus is a plug flow reaction (PFR) comprising two or more Raman sensors configured to obtain Raman spectra from a solution flowing through the PFR at two or more points separated by a predetermined distance. In some embodiments, the continuous IVT reaction apparatus is a plug flow reaction (PFR) in which the Raman sensor is configured to obtain a Raman spectrum from an output end of the continuous IVT reaction apparatus.

In some embodiments, the continuous IVT reaction apparatus further comprises an mRNA purification module. In some embodiments, the mRNA purification module is selected from (a) a tangential flow filtration module; (b) an oligo-dT chromatography module; and/or (c) a high performance liquid chromatography (HPLC) module.

In some embodiments, a reaction apparatus comprises a plug flow reactor. A plug flow reactor (PFR), also known in the art as a continuous tubular reactor (CTR), is a tubular reactor where the feed is continuously introduced at one end and the products are continuously removed from the other end. A plug flow reactor is not mixed longitudinally along the length of the reactor. Accordingly, as a reaction mixture flows through the reactor, the longitudinal position of a unit of reaction mixture indicates the time for which the unit has been reacting. The residence time for a reaction mixture in a PFR is thus the time taken for a unit of the reaction mixture to reach the outlet location after being input into the PFR.

Feed solutions may be introduced to a PFR by any suitable method. Namely, pumps are used to control the flow rate of the system. In a PFR system, the inlet flow rate may be the same as the outlet flow rate to avoid a buildup or drop in pressure. The input flow rate may be controlled by positioning an inlet valve to achieve a desired the input flow rate.

Residence time may be a function of input and output flow rates and the total volume of the reactor. Accordingly, the length and cross-sectional area of the PFR are relevant in determining the residence time of a given PFR system. Because the cross-sectional area of a PFR is typically held constant, the length of the PFR and the flow rate may be the variables changed to control the flow rate. In some embodiments, the product flow rate may have a set point, and thus the flow rate of the reaction mixture is held constant, but the length of the PFR may be modified to alter the residence time of the IVT reaction mixture. The active length of the PFR may be changed by opening and/or closing valves change the outlet location (position at which IVT reaction mixture is output) of the PFR.

In some embodiments, residence time of an IVT reaction mixture in the PFR is decreased by opening a valve upstream of a current outlet location, after it has been determined that the target endpoint occurred prior to the IVT reaction mixture reaching an end of the active length of the PFR.

In some embodiments, residence time of an IVT reaction mixture is increased by closing an outlet and opening a valve downstream of the outlet, after it has been determined that the target endpoint did not occur prior to the IVT reaction mixture reaching an end of the active length of the PFR.

A PFR system may be modeled using the following design equation.

$$V = {F}_{A 0} {\int }_{0}^{X} \frac{dX}{- {r}_{A}} \qquad Eq . 3$$

Where;
V: Volume
F_{A0}: Inlet molar flow rate of reactant
X: Conversion
r_{A}: Reaction rate

Mathematical modeling may be used, such as Equation 3, to determine a suitable volume to achieve the desired residence time.

In some embodiments, a heat exchanger is present on the exterior surface of the reactor. Heat exchange may be used to maintain the reactor (and reaction mixture) at a desired temperature.

The PFR may include materials and components for maintaining desired flow conditions (e.g., input feed rate and/or output flow rate, friction, pressure drop, turbulence). For example, a PFR may include pipe material that applies minimal or no access friction to an IVT reaction mixture. Such access friction may be avoided to limit a pressure drop and/or turbulence that may reduce the efficiency of an IVT reaction. As another example, pipe dimensions may be adjusted, such as by increasing diameter to reduce surface contact and friction. These and other characteristics for determining suitability for an IVT reaction, such as resistance to corrosion, may determined by the skilled artisan.

The aforementioned valves may be diaphragm valves, solenoid valves, pinch valves, diverting valves, or any other valve with sufficient pressure ratings. These valves may be used in any combination in order to obtain the active length adjustability described above.

In some embodiments, a reaction apparatus comprises a continuous stir tank reactor (CSTR). A CSTR is characterized by a continuous flow of reactants into and products out of the reaction system. A feature of a CSTR is that the concentration of reactants and temperature are approximately equal throughout due to an impeller gently stirring the contents of the system. This results in the product stream being similar to the contents of the system.

The residence time in a CSTR can be changed by creating changes in the inlet flow rate and then returning the inlet flow rate to its original value. As mentioned above, the volume of contents in the system along with the flow rate of the system dictate the residence time. During normal operation, the inlet and outlet flow rates are equal, keeping the total volume constant. However, if it is desired for the volume to change to modify the residence time, the inlet flow rate may temporarily be changed. If the inlet flow rate in decreased for a set period of time, the comparatively larger outlet flow rate will start to decrease the volume of the system. If the inlet flowrate is increases for a set period of time, the comparatively smaller outlet flow rate will start to increase the volume of the system. The skilled artisan will appreciate that a suitable CSTR will have a volume sufficient to permit variations in the volume of an IVT reaction mixture caused by adjusting input feed rate(s) and output flow rate.

A CSTR system may be modeled using the following design equation.

$$V = \frac{{F}_{A 0} X}{- {R}_{A}} \qquad Eq . 4$$

Where;
V: Volume
F_{A0}; Inlet molar flow rate of reactant
X: Conversion
r_{A}: Reaction rate

Mathematical modeling may be conducted, e.g., using Equation 4, to extrapolate a volume needed in order to achieve the desired residence time, following determination of reaction rate, target endpoint, and target residence time based on Raman spectra collected.

A heat exchanger may be present on the exterior surface of the CSTR to allow maintenance of the reactor (and IVT reaction mixture) at a desired temperature. A CSTR may be made from any suitable material, such as one that does not corrode after exposure to, or otherwise react with, the IVT reaction mixture. These and other characteristics for determining suitability for an IVT reaction within ordinary skill in the art.

The PFR may include materials and components for maintaining desired flow conditions (e.g., input feed rate and/or output flow rate, friction, pressure drop, turbulence). For example, a PFR may include pipe material that applies minimal or no access friction to an IVT reaction mixture. Such access friction may be avoided to limit a pressure drop and/or turbulence that may reduce the efficiency of an IVT reaction. As another example, pipe dimensions may be adjusted, such as by increasing diameter to reduce surface contact and friction.

### DNase Digestion

In some embodiments, an IVT reaction mixture output from a first reaction apparatus (e.g., CSTR or PFR) flows into an additional reaction apparatus, where DNA in the IVT reaction mixture is digested by a DNase. In some embodiments, a continuous reaction apparatus further comprises a DNase reaction apparatus (a) fluidically coupled downstream of the continuous IVT reaction apparatus, and (b) configured to receive a third feed solution comprising a DNase, the DNase reaction apparatus comprising a continuous plug flow reaction (CPFR) comprising one or more curved pipes.

Digestion of DNA, such as a full-length template used for transcription, by DNAse produces multiple smaller DNA fragments, which are more easily separated from full-length RNAs, allowing for removal of such DNA contaminants from a mixture containing desired RNA. For example, DNA fragments, being smaller than RNA transcripts, may pass through pores of a tangential flow filtration (TFF) membrane while larger RNAs do not, and may thus be removed by TFF. As another example, DNA fragments may traverse a reverse phase chromatography column at a different rate than RNAs, particularly mRNAs having a hydrophobic polyA tail, and may thus be removed by reverse phase HPLC.

In some embodiments, an additional feed solution comprising the DNase and an additional buffer is introduced into the additional reaction apparatus, such that the additional reaction apparatus contains a DNase reaction mixture.

In some embodiments, the IVT reaction mixture flows continuously into the additional reaction apparatus. In some embodiments, the output flow rate of the IVT reaction mixture from the first continuous reaction apparatus (where IVT occurs) is substantially similar to the rate at which the IVT reaction mixture flows into the additional reaction apparatus. In some embodiments, the output flow rate of the IVT reaction mixture from the first continuous reaction apparatus (where IVT occurs) is substantially similar to the rate at which the DNasae reaction mixture flows through the additional reaction apparatus. In some embodiments, the output flow rate of the IVT reaction mixture from the first continuous reaction apparatus (where IVT occurs) is substantially similar to the rate at which the IVT reaction mixture flows into the additional reaction apparatus, the DNase reaction mixture flows through the additional reaction apparatus, and the DNase reaction mixture flows out of the additional reaction apparatus.

In some embodiments, the additional reaction apparatus for DNase digestion is a continuous plug flow reaction (CPFR) having one or more curved pipes. The curved pipes may have any suitable diameter. In some embodiments, each curved pipe has a substantially equal diameter.

In some embodiments, the DNase reaction mixture flows through the CPFR with a Dean number (De) of at least 30. Any suitable method may be used to determine the Dean number of flow through the CPFR, such as mathematical modeling. See, e.g., Zhang, Pusheng & Gros, Y & Roberts, R & Benard, Andre. (2010). Modeling of Turbulent Flow with Particle Deposition in Curved Pipes. In some embodiments, the Dean number is at least 20, at least 25, at least 30, at least 40, at least 50, or at least 60. In some embodiments, the Dean number is 30-100, 30-75, 30-60, 30-45, or 30-40.

In some embodiments, each of the curved pipes has a radius of curvature that is 180% to 400% of the diameter of the curved pipe. Methods of determining a curved pipe's radius of curvature are known in the art. In some embodiments, each curved pipe has a radius of curvature that is 180% to 350%, 180% to 300%, 180% to 250%, or 180% to 225% of the curved pipe's diameter.

In some embodiments, the flow of the DNase reaction mixture through the CPFR has a pressure drop of 0.5 bar or less. Any suitable method may be used to calculate pressure drop, such as those known in the art. See, e.g., Keulegan and Beij, J Res Natl Inst Stand Technol. 1937. 18:89-114.

In some embodiments, the DNase reaction mixture is incubated in the additional reaction apparatus, such that the DNase cleaves one or more DNAs to produce one or more DNA fragments. Any suitable DNase may be used for DNase digestion, such as DNase I-XT. In some embodiments, the DNase cleaves double-stranded DNA. In some embodiments, the digestion of DNA by the DNase generates oligonucleotides no longer than 100 nucleotides, no longer than 80 nucleotides, no longer than 60 nucleotides, no longer than 50 nucleotides, no longer than 40 nucleotides, no longer than 30 nucleotides, no longer than 20 nucleotides, or no longer than 10 nucleotides in length. In some embodiments, at least 70%, at least 80%, at least 90%, at least 95%, at least 96%, at least 97%, at least 98%, at least 99%, or up to 100% of DNA molecules in the DNase reaction mixture are 100 nucleotides long or shorter after DNAse digestion.

In some embodiments, the DNase reaction mixture flows continuously from the additional reaction apparatus to an mRNA purification module. In some embodiments, the output flow rate of the DNase reaction mixture from the additional reaction apparatus (where DNase digestion occurs) is substantially similar to the rate at which the DNase reaction mixture flows into the mRNA purification module. In some embodiments, the output flow rate of the DNase reaction mixture from the additional reaction apparatus (where DNase digestion occurs) is substantially similar to the rate at which the DNase reaction mixture flows through the mRNA purification module.

The mRNA purification module may include an apparatus for purifying mRNA by any suitable method, such as tangential flow filtration, oligo-dT chromatography, and/or reverse phase HPLC. In some embodiments, DNA fragments are removed from the mRNA by TFF. In some embodiments, the mRNA is separated from one or more impurities other than DNA fragments by oligo-dT chromatography. In some embodiments, the mRNA is separated from one or more impurities other than DNA fragments by reverse phase HPLC. The one or more other impurities separated from the mRNA may be, for instance, NTPs, cap analogs, RNA polymerases, abortive transcripts, dsRNAs, and/or buffer components. In some embodiments, the mRNA is separated from DNA fragments by TFF, and followed by introducing the mRNA into an oligo-dT chromatography module. These and other purification methods are described below in the section “mRNA purification.”

In some embodiments, a composition comprising mRNA that is isolated by the mRNA purification module(s) following DNase digestion comprises 0.1% or less, 0.01% or less, 0.001% or less, or 0.0001% or less DNA by weight (% wt/wt).

### In Vitro Transcription (IVT)

In vitro transcription of RNA is known in the art and is described in International Publication No. WO 2014/152027, which is incorporated by reference herein to the extent it discloses IVT methods. In some embodiments, the mRNA is prepared in accordance with any one or more of the methods described in International Publication Nos. WO 2018/053209 and WO 2019/036682, each of which is incorporated by reference herein to the extent they disclose RNA production methods.

### DNA

Any suitable DNA molecule may be used as a template for in vitro transcription. DNA templates for IVT generally include a promoter that an RNA polymerase uses to initiate transcription. In some embodiments, the DNA comprises an RNA polymerase promoter located 5′ to and operably linked to the nucleotide sequence to be transcribed. In some embodiments, the DNA encodes, in 5′-to-3′ order: a 5′ untranslated region, an open reading frame followed by one or more stop codons, a 3′ untranslated region, and a polyA tail.

In some embodiments, a DNA template used in IVT is a plasmid. In some embodiments, the plasmid is linearized. DNA templates such as plasmids may be generated by any suitable method, such as introduction of the plasmid into a host cell (e.g., bacterial cells, e.g., E. coli, e.g., DH-1 cells) and allowing the host cell to amplify the plasmid, with such amplified plasmids being isolated from host cells and purified. Starting material for amplification may be manufactured by any suitable method, such as molecular cloning or de novo synthesis of a desired DNA sequence.

### RNA Polymerases

Any suitable RNA polymerase may be used for in vitro transcription. Non-limiting examples of RNA polymerases include those of bacteriophages such as T7, T3, SP6, and K11. In some embodiments, the RNA polymerase is selected from the group consisting of T7, T3, SP6, and K11 RNA polymerase. In some embodiments, the RNA polymerase is a T7 RNA polymerase. In some embodiments, the RNA polymerase initiates with a guanosine nucleotide (e.g., GTP or GDP).

In some embodiments the RNA polymerase is a wild-type RNA polymerase. In some embodiments, the RNA polymerase is an RNA polymerase variant, such as those described in WO 2020/172239, incorporated herein by reference to the extent it describes RNA polymerase variants. RNA polymerase variants may include at least one amino acid substitution, relative to the wild-type (WT) RNA polymerase. A WT T7 RNA polymerase is represented by SEQ ID NO: 45. In some embodiments, the RNA polymerase is a variant RNA polymerase comprising the amino acid sequence of any one of SEQ ID NOs: 46-49.

### Nucleotides

An in vitro transcription reaction mixture generally comprises nucleotide triphosphates (NTPs), which an RNA polymerase incorporates into an RNA transcript. Nucleotide diphosphates (NDPs) and monophosphates (NMPs) may initiate the reaction, but elongation (incorporation of the second nucleotide through the last nucleotide) requires nucleotide triphosphates. NTPs, NDPs, and NMPs may be manufactured by chemical synthesis, or purchased from commercial sources. Nucleotides may be modified, unmodified, or a mixture of both modified and unmodified nucleotides (e.g., some portion of adenosine nucleotides are modified ATP and some portion are unmodified ATP).

In some embodiments, an IVT reaction mixture comprises unmodified ATP. In some embodiments the IVT reaction mixture does not comprise modified ATP.

In some embodiments, the IVT reaction mixture comprises modified ATP. In some embodiments, the IVT reaction mixture does not comprise unmodified ATP. In some embodiments, 100% of adenine nucleotides in the IVT reaction mixture are modified ATPs. In some embodiments, the modified ATP comprises a modified nucleobase selected from digoxigeninated adenine, N6-methyladenine, 7-deazaadenine, 7-deaza-7-propargylaminoadenine, 8-azaadenine, 8-azidoadenine, 8-chloroadenine, 8-oxoadenine, araadenine, N1-methyladenine, N6-methyladenine, 3-deazaadenine, 2,6-diaminoadenine, 2-methyl-thio-N6-isopentenyladenine (ms2i6A), 2-methylthio-N6-methyladenine (ms2m6A), N6-(cis-hydroxyisopentenyl) adenine (io6A), 2-methylthio-N6-(cis-hydroxyisopentenyl) adenine (ms2io6A), N6-glycinylcarbamoyladenine (g6A), N6-threonylcarbamoyladenine (t6A), 2-methylthio-N6-threonyl carbamoyladenine (ms2t6A), N6-methyl-N6-threonylcarbamoyladenine (m6t6A), N6-hydroxynorvalylcarbamoyladenine (hn6A), 2-methylthio-N6-hydroxynorvalyl carbamoyladenine (ms2hn6A), N6,N6-dimethyladenine (m62A), and N6-acetyladenine (ac6A).

In some embodiments, the modified ATP is N6-methyladenosine triphosphate.

In some embodiments, an IVT reaction mixture comprises unmodified CTP. In some embodiments the IVT reaction mixture does not comprise modified CTP.

In some embodiments, the IVT reaction mixture comprises modified CTP. In some embodiments, the IVT reaction mixture does not comprise unmodified CTP. In some embodiments, 100% of cytosine nucleotides in the IVT reaction mixture are modified CTPs. In some embodiments, the modified CTP comprises a modified nucleobase selected from digoxigeninated cytosine, 2-thiocytosine, 5-aminoallylcytosine, 5-bromocytosine, 5-carboxycytosine, 5-formylcytosine, 5-hydroxycytosine, 5-hydroxymethylcytosine, 5-methoxycytosine, 5-methylcytosine, 5-propargylaminocytosine, 5-propynylcytosine, 6-azacytosine, aracytosine, cyanine 3-5-propargylaminocytosine, cyanine 3-aminoallylcytosine, cyanine 5-6-propargylaminocytosine, cyanine 5-aminoallylcytosine, desthiobiotin-6-aminoallylcytosine, N4-biotin-OBEA-cytosine, N4-methylcytosine, pseudoisocytosine, and thienocytosine. In some embodiments, the modified CTP is 5-methylcytidine triphosphate.

In some embodiments, an IVT reaction mixture comprises unmodified GTP. In some embodiments the IVT reaction mixture does not comprise modified GTP.

In some embodiments, the IVT reaction mixture comprises modified GTP. In some embodiments, the IVT reaction mixture does not comprise unmodified GTP. In some embodiments, 100% of guanosine nucleotides in the IVT reaction mixture are modified GTPs. In some embodiments, the modified GTP comprises a modified nucleobase selected from digoxigeninated guanine, 6-thioguanine, 7-deazaguanine, 7-deaza-7-propargylaminoguanine, 8-oxoguanine, araguanine, biotin-16-7-deaza-7-propargylaminoguanine, isoguanine, N2-methylguanine, O6-methylguanine, thienoguanine, and 2,6-daminoguanine.

In some embodiments, an IVT reaction mixture comprises unmodified UTP. In some embodiments the IVT reaction mixture does not comprise modified UTP.

In some embodiments, the IVT reaction mixture comprises modified UTP. In some embodiments, the IVT reaction mixture does not comprise unmodified UTP. In some embodiments, 100% of uracil nucleotides in the IVT reaction mixture are modified UTP. In some embodiments, the modified UTP comprises a modified nucleobase selected from pseudouracil (ψ), N1-methylpseudouracil (mlψ), 1-ethylpseudouracil, 2-thiouracil, 4′-thiouracil, 2-thio-1-methyl-1-deaza-pseudouracil, 2-thio-1-methyl-pseudouracil, 2-thio-5-aza-uracil, 2-thio-dihydropseudouracil, 2-thio-dihydrouracil, 2-thio-pseudouracil, 4-methoxy-2-thio-pseudouracil, 4-methoxy-pseudouracil, 4-thio-1-methyl-pseudouracil, 4-thio-pseudouracil, 5-aza-uracil, dihydropseudouracil, 5-methyluracil, 5-methoxyuracil (mo5U) and 2′-O-methyluracil. In some embodiments, the modified UTP is N1-methylpseudouridine triphosphate.

In some embodiments, the IVT reaction mixture comprises modified NTPs comprising a modified sugar. In some embodiments, the modified NTPs comprise a modified sugar selected from 2′-thioribose, 2′,3′-dideoxyribose, 2′-amino-2′-deoxyribose, 2′ deoxyribose, 2′-azido-2′-deoxyribose, 2′-fluoro-2′-deoxyribose, 2′-O-methylribose, 2′-O-methyldeoxyribose, 3′-amino-2′,3′-dideoxyribose, 3′-azido-2′,3′-dideoxyribose, 3′-deoxyribose, 3′-O-(2-nitrobenzyl)-2′-deoxyribose, 3′-O-methylribose, 5′-aminoribose, 5′-thioribose, 5-nitro-1-indolyl-2′-deoxyribose, 5′-biotin-ribose, 2′-O,4′-C-methylene-linked, 2′-O,4′-C-amino-linked ribose, and 2′-O,4′-C-thio-linked ribose.

In some embodiments, the IVT reaction mixture comprises modified NTPs comprising one or more modified phosphates. In some embodiments, the modified NTPs comprise a modified phosphate selected from phosphorothioate (PS), thiophosphate, 5′-O-methylphosphonate, 3′-O-methylphosphonate, 5′-hydroxyphosphonate, hydroxyphosphanate, phosphoroselenoate, selenophosphate, phosphoramidate, carbophosphonate, methylphosphonate, phenylphosphonate, ethylphosphonate, H-phosphonate, guanidinium ring, triazole ring, boranophosphate (BP), methylphosphonate, and guanidinopropyl phosphoramidate.

### Caps and Cap Analogs

In some embodiments, an IVT reaction mixture comprises a cap or cap analog, which is incorporated into the 5′ end of an mRNA during IVT. A cap analog may be, for example, a dinucleotide cap, a trinucleotide cap, or a tetranucleotide cap. In some embodiments, a cap analog is a dinucleotide cap. In some embodiments, a cap analog is a trinucleotide cap. In some embodiments, a cap analog is a tetranucleotide cap.

5′-capping of polynucleotides may be completed concomitantly during an in vitro transcription reaction using, for example, the following chemical RNA cap analogs to generate the 5′-guanosine cap structure according to manufacturer protocols: 3′-O-Me-m7G(5′)ppp(5′)G [the ARCA cap]; G(5′)ppp(5′)A; G(5′)ppp(5′)G; m7G(5′)ppp(5′)A; m7G(5′)ppp(5′)G (New England BioLabs, Ipswich, MA). 5′-capping of modified mRNA may be completed post-transcriptionally using, for example, a Vaccinia Virus Capping Enzyme to generate the “Cap 0” structure: m7G(5′)ppp(5′)G (New England BioLabs, Ipswich, MA). A Cap 1 structure may be generated using both Vaccinia Virus Capping Enzyme and a 2′-O methyl-transferase to generate: m7G(5′)ppp(5′)G-2′-O-methyl. A Cap 2 structure may be generated from the Cap 1 structure followed by the 2′-O-methylation of the 5′-antepenultimate nucleotide using a 2′-O methyl-transferase. A Cap 3 structure may be generated from the Cap 2 structure followed by the 2′-O-methylation of the 5′-preantepenultimate nucleotide using a 2′-O methyl-transferase. Enzymes may be derived from a recombinant source. Other cap analogs, such as a 7 mG(5′)ppp(5′)NlmpNp cap, may be used.

### Buffer

IVT reaction mixtures may comprise a buffer, e.g., Tris, phosphate, or Good's buffer. The concentration of a buffer in an IVT reaction mixture may be, for example, at least 10 mM, at least 20 mM, at least 30 mM, at least 40 mM, at least 50 mM, at least 60 mM, at least 70 mM, at least 80 mM, at least 90 mM, at least 100 mM or at least 110 mM phosphate. In some embodiments, the concentration of phosphate is 20-60 mM or 10-100 mM. In some embodiments, the buffer comprises Tris-HCl. For example, the buffer may comprise 10-100 mM, 10-80 mM, 10-60 mM, 20-100 mM, 20-18 mM, 20-60 mM Tris-HCl. In some embodiments, the buffer comprises 40 mM Tris-HCl.

### Magnesium

IVT reaction mixtures may comprise magnesium. In some embodiments, the IVT reaction mixture comprises Mg(OAc)₂. In some embodiments, the molar ratio of magnesium ions to NTPs is 1:1, 1:2, 1:3, 1:4 or 1:5. In some embodiments, the molar ratio of magnesium ions to NTPs is 1:1 to 1:5.

mRNA Purification

In some embodiments, mRNA is separated from one or more other components of a mixture (e.g., IVT reaction mixture or DNase reaction mixture) using an mRNA purification module. In some embodiments, an apparatus comprises an mRNA purification module. Non-limiting examples of mRNA purification methods and associated modules include tangential flow filtration (TFF), oligo-dT chromatography, and high performance liquid chromatography (HPLC).

### Tangential Flow Filtration (TFF)

In some embodiments, mRNA is separated from one or more other components of a mixture by tangential flow filtration (TFF). In some embodiments, a reaction apparatus comprises a TFF mRNA purification module.

In TFF, a composition containing mRNA flows over a filtration membrane (TFF membrane) comprising pores, with the pores of the membrane being oriented perpendicular to the direction of flow. Components of the composition flow through the pores, if able, while components that do not pass through the pores are retained in the composition. TFF thus removes smaller impurities, such as peptide fragments, amino acids, DNA fragments, and nucleotides (e.g., NTPs) from a mixture, while larger molecules, such as full-length mRNAs, are retained in the mixture. Additionally, RNA polymerases may produce double-stranded RNA transcripts during IVT, comprising an RNA:RNA hybrid of a full-length RNA transcript and another RNA with a complementary sequence. The second RNA that is hybridized to the full-length RNA transcript may be another full-length RNA, or a smaller RNA that hybridizes to only a portion of the full-length transcript. Like DNA fragments produced by DNase digestion of DNA templates, these small RNAs may also be removed during TFF, so that fewer dsRNA molecules are present in the filtered composition.

The size of the pores of the TFF membrane affect which components are filtered (removed) out and which are retained in the mixture. Generally, TFF membranes are characterized in terms of a molecular weight cutoff, with components smaller than the molecular weight cutoff being removed from the mixture during TFF, while components larger than the molecular weight cutoff being retained in the mixture. In some embodiments, the tangential flow filtration comprises using a TFF membrane with a molecular weight cutoff of 500 kDa or less, 200 kDa or less, 150 kDa or less, 100 kDa or less, 50 kDa or less, 40 kDa or less, 30 kDa or less, 20 kDa or less, or lower.

TFF methods are described in International Application No. PCT/US2023/012261, which is incorporated by reference herein for this purpose.

### Oligo-dT Chromatography

In some embodiments, mRNA is separated from one or more other components of a mixture by oligo-dT chromatography. In some embodiments, mRNA is separated by multicolumn oligo-dT chromatography. In some embodiments, a reaction apparatus comprises an oligo-dT mRNA purification module. In some embodiments, a reaction apparatus comprises a multicolumn oligo-dT mRNA purification module.

Oligo-dT refers to a DNA oligonucleotide comprising multiple repeated thymidine bases. This sequence of repeated thymidine bases bind to the polyA tail of mRNAs. Immobilization of oligo-dT by bonding (e.g., covalent bonding) to particles of the stationary phase promotes binding of mRNAs to the stationary phase. mRNAs bound to oligo-dT may be retained on the stationary phase while other components of the mixture (e.g., amino acids, peptide fragments, nucleotides, DNA fragments) are removed. Non-mRNA components may be removed by any suitable method, such as passage of another mobile phase (e.g., washing solution) over the stationary phase, which has higher affinity for impurities than for mRNAs bound to the oligo-dT. After impurities are removed, the mRNA may be released from the stationary phase using another mobile phase (e.g., elution buffer) that separates oligo-dT-bound mRNAs from the stationary phase. Oligo-dT chromatography is described in International Application Nos. PCT/US2020/046069 and PCT/US2022/040139, which are incorporated by reference herein for this purpose.

### High Performance Liquid Chromatograph (HPLC) Purification

In some embodiments, mRNA is separated from one or more other components of a mixture by HPLC. In some embodiments, a reaction apparatus comprises an HPLC mRNA purification module.

HPLC separates molecules of a mixture (e.g., mRNAs of a reaction mixture) from one or more other components (e.g., dsRNA contaminants, abortive transcripts, and DNA templates) of the mixture using a mobile phase and stationary phase. Typically, the mixture is dispersed in a mobile phase, which is passed over a stationary phase of a column, and components of the mixture traverse the column at different rates based on differential affinity for the stationary phase, allowing separation of a desired component from others. The composition of stationary and mobile phases may vary to separate a desired component (e.g., mRNA) from others, and elute that component from the column to obtain a composition having increased purity with respect to that component. HPLC is described in International Application Nos. PCT/US2018/046990, PCT/US2018/046993, and PCT/US2022/039037; and U.S. Pat. No. 8,383,340, which are incorporated by reference herein for this purpose.

### Stationary Phases

In some embodiments, the stationary phase comprises fiber, particles, resin, and/or beads, Examples of stationary phases include but are not limited to resin, silica (e.g., alkylated and non-alkylated silica), polystyrenes (e.g., alkylated and non-alkylated polystyrenes), polystyrene divinylbenzenes, etc. In some embodiments, a stationary phase comprises particles, for example porous particles. In some embodiments, a stationary phase (e.g., particles of a stationary phase) is hydrophobic (e.g., made of an intrinsically hydrophobic material, such as polystyrene divinylbenzene), or comprise hydrophobic functional groups. In some embodiments, a stationary phase is a membrane or monolithic stationary phase. A monolithic stationary phase is a continuous, unitary, porous structure prepared by in situ polymerization or consolidation inside the column tubing. In some embodiments, the surface is functionalized to convert it into a sorbent with the desired chromatographic binding properties.

The particle size (e.g., as measured by the diameter of the particle) of a stationary phase of a column can vary. In some embodiments, the particle size of the stationary phase ranges from about 1 μm to about 100 μm (e.g., any value between 1 and 100, inclusive) in diameter. In some embodiments, the particle size of the column stationary phase ranges from about 2 μm to about 10 μm, about 2 μm to about 6 μm, or about 4 μm in diameter. The pore size of particles (e.g., as measured by the diameter of the pore) can also vary. In some embodiments, the particles comprise pores having a diameter of about 500 Å to about 5000 Å, about 800 Å to about 3000 Å, or about 1000 Å to about 2000 Å.

The temperature of the stationary phase (e.g., the stationary phase within the column during purification) can vary. In some embodiments, the stationary phase has a temperature from about 4° C. to about 99° C. (e.g., any temperature between 4° C. and 99° C.). In some embodiments, the stationary phase has a temperature from about 4° C. to about 40° C. (e.g., any temperature between 4° C. and 40° C., for example about 4° C., about 10° C., about 20° C., about 25° C., about 30° C., about 35° C., or about 40° C.). In some embodiments, the stationary phase has a temperature from about 20° C. to about 40° C. (e.g., any temperature between 20° C. and 40° C.).

In some embodiments, a stationary phase is comprised in a hollow fiber membrane. A hollow fiber membrane (HFM) refers to a hollow cylinder, with the walls of the cylinder comprising a fibrous membrane. The walls of the hollow fiber membrane may comprise a stationary phase, such as oligo-dT resin or beads, that allows for binding of a desired molecule, such as an mRNA. A solution containing the desired molecule may then be passed through the hollow center of the hollow fiber membrane, allowing the desired molecule to be retained, followed by one or more washing and/or eluting steps to separate the desired molecule from any impurities. In this manner, the walls of the membrane function as the stationary phase of the chromatography column, as an alternative to a particulate stationary phase that is packed into the interior space of a chromatography column. However, the empty space within the center of the hollow fiber membrane allows solutions to be passed through at greater pressures than are typically feasible with a packed chromatography column. Hollow fiber membranes may be used as an alternative to a stationary phase packed into the interior of the chromatography column, or the interior of a hollow fiber membrane may be packed with a particulate stationary phase, such as resin or beads, allowing both the packed stationary phase and the walls of the membrane to retain a desired molecule. Hollow fiber membranes may comprise one or more suitable stationary phases, such as a stationary phase of a reverse phase chromatography column or oligo-dT. In some embodiments, the hollow fiber membrane comprises oligo-dT.

In some embodiments, a stationary phase is comprised in a sheet membrane. In contrast to cylindrical hollow fiber membranes, which contain a hollow center through which a solution is passed, a solution is applied to one side of a sheet, and exits the other side after passing through one or more sheets. In some embodiments, a sheet membrane comprises a single flat sheet. In some embodiments, a sheet membrane comprises a sheet wound into a spiral. In some embodiments, a sheet membrane comprises multiple sheets. Sheet membranes may comprise one or more suitable stationary phases, such as a stationary phase of a reverse phase chromatography column or oligo-dT. In some embodiments, the sheet membrane comprises oligo-dT.

### Mobile Phases

In some embodiments of mRNA purification methods, a mobile phase comprises Tris and/or chelator, such as EDTA (e.g., Tris-EDTA, also referred to as TAE). As used herein, a “mobile phase” is an aqueous solution comprising water and/or one or more organic solvents used to carry an analyte (or analytes), such as a nucleic acid (e.g., mRNA) or mixture of nucleic acids (e.g., mRNAs) through a column.

In some embodiments, a mobile phase comprises a polar organic solvent. Examples of polar organic solvents suitable for inclusion in a mobile phase include but are not limited to alcohols, ketones, nitrates, esters, amides and alkylsulfoxides. In some embodiments, a mobile phase comprises one or more organic solvents selected from the group consisting of acetonitrile, methanol, ethanol, propanol, isopropanol, dimethylformamide, methyl acetate, acetone, and dimethyl sulfoxide (DMSO), hexaline glycol, polar aprotic solvents (including, e.g., tetrahydrofuran (THF), dimethyl sulfoxide (DMSO), dimethylformamide (DMF), acetonitrile, acetone, etc.), C₁₋₄ alkanols, C₁₋₆ alkandiols, and C₂₋₄ alkanoic acids. The concentration of organic solvent in a mobile phase can vary. For example, in some embodiments, the volume percentage (v/v) of an organic solvent in a mobile phase varies from 0% (absent) to about 100% of a mobile phase. In some embodiments, the volume percentage of organic solvent in a mobile phase is between about 5% and about 75% v/v. In some embodiments, the volume percentage of organic solvent in a mobile phase is between about 25% and about 60% v/v. In some embodiments, the concentration of organic solvent in a mobile phase is about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% v/v. In some embodiments, a mobile phase comprises acetonitrile. In some embodiments, a mobile phase comprises additional components, for example as described in U.S. Patent Publication US 2005/0011836, which is incorporated by reference herein for this purpose.

In some embodiments, one or more solvent solutions (e.g., 1, 2, 3, 4, 5, or more) of the mobile phase comprise a combination of at least two ion pairing agents (e.g., 2, 3, 4, 5, or more). As used herein, an “ion pairing agent” or an “ion pair” refers to an agent (e.g., a small molecule) that functions as a counter ion to a charged (e.g., ionized or ionizable) functional group on an HPLC analyte (e.g., a nucleic acid) and thereby changes the retention time of the analyte as it moves through the stationary phase of an HPLC column. Generally, ion paring agents are classified as cationic ion pairing agents (which interact with negatively charged functional groups) or anionic ion pairing agents (which interact with positively charged functional groups). The terms “ion pairing agent” and “ion pair” further encompass an associated counter-ion (e.g., acetate, phosphate, bicarbonate, bromide, chloride, citrate, nitrate, nitrite, oxide, sulfate and the like, for cationic ion pairing agents, and sodium, calcium, and the like, for anionic ion pairing agents). In some embodiments, one or more ion pairing agents utilized in the methods described by the disclosure is a cationic ion pairing agent. Examples of cationic ion pairing agents include but are not limited to certain protonated or quaternary amines (including e.g., primary, secondary and tertiary amines) and salts thereof, such as a trietheylammonium salt (e.g., triethylammonium acetate (TEAA)), a tributylammonium salt (e.g., tetrabutylammonium phosphate (TBAP) or tetrabutylammonium chloride (TBAC)), a hexylammonium salt (e.g., hexylammonium acetate (HAA)), a dibutylammonium salt (e.g., dibutylammonium acetate (DBAA)), a tetrapropylammonium salt (e.g., tetrapropylammonium bromide (TPAB)), a dodecyltrimethylammonium salt (e.g., dodecyltrimethylammonium chloride (DTMAC)), or a tetra(decyl)ammonium salt (e.g., tetra(decyl)ammonium bromide (TDAB)), a dihexylammonium salt (e.g., dihexylammonium acetate (DHAA)), a dipropylammonium salt (e.g., dipropylammonium acetate (DPAA)), a myristyltrimethylammonium salt (e.g., myristyltrimethylammonium bromide (MTEAB)), a tetraethylammonium salt (e.g., tetraethylammonium bromide (TEAB)), a tetraheptylammonium salt (e.g., tetraheptylammonium bromide (THepAB)), a tetrahexylammonium salt (e.g., tetrahexylammonium bromide (THexAB)), a tetrakis(decyl)ammonium salt (e.g., tetrakis(decyl)ammonium bromide (TrDAB)), a tetramethylammonium salt (e.g., tetramethylammonium bromide (TMAB)), a tetraoctylammonium salt (e.g., tetraoctylammonium bromide (TOAB)), or a tetrapentylammonium salt (e.g., tetrapentylammonium bromide (TPeAB)). In some embodiments, one or more solvent solutions of the mobile phase comprise a combination of two or more ion pairing agents selected from the group consisting of a trietheylammonium salt, tributylammonium salt, hexylammonium salt, dibutylammonium salt, tetrapropylammonium salt, dodecyltrimethylammonium salt, tetra(decyl)ammonium salt, dihexylammonium salt, dipropylammonium salt, myristyltrimethylammonium salt, tetraethylammonium salt, tetraheptylammonium salt, tetrahexylammonium salt, tetrakis(decyl)ammonium salt, tetramethylammonium salt, tetraoctylammonium salt, and tetrapentylammonium salt. In some embodiments, one or more solvent solutions of the mobile phase comprise a combination of two or more ion pairing agents selected from the group consisting of HAA, TBAP, TPAB, TBAC, DBAA, TEAA, DTMAC, TDAB, DHAA, DPAA MTEAB, TEAB, THepAB, THexAB, TrDAB, TMAB, TOAB, and TPeAB. In some embodiments, one or more solvent solutions of the mobile phase comprise a combination of (i) TPAB and TBAC, (ii) DBAA and TEAA, or (iii) TBAP and TEAA. In some embodiments, one or more solvent solutions of the mobile phase comprise a combination of TPAB and TBAC.

In some embodiments, one or more solvent solutions (e.g., 1, 2, 3, 4, 5, or more) of the mobile phase comprise a single ion pairing agent. In some embodiments, one or more ion pairing agents utilized in the methods described by the disclosure is a cationic ion pairing agent. In some embodiments, the ion pairing agent is a cationic ion pairing agent. In some embodiments, one or more solvent solutions of the mobile phase comprise a salt selected from the group consisting of a trietheylammonium salt, tributylammonium salt, hexylammonium salt, dibutylammonium salt, tetrapropylammonium salt, dodecyltrimethylammonium salt, tetra(decyl)ammonium salt, dihexylammonium salt, dipropylammonium salt, myristyltrimethylammonium salt, tetraethylammonium salt, tetraheptylammonium salt, tetrahexylammonium salt, tetrakis(decyl)ammonium salt, tetramethylammonium salt, tetraoctylammonium salt, and tetrapentylammonium salt. In some embodiments, one or more solvent solutions of the mobile phase comprise HAA, TBAP, TPAB, TBAC, DBAA, TEAA, DTMAC, TDAB, DHAA, DPAA MTEAB, TEAB, THepAB, THexAB, TrDAB, TMAB, TOAB, TPeABHAA, TBAP, TPAB, TBAC, DBAA, TEAA, DTMAC, or TDAB. In some embodiments, each of one or more solvents of the mobile phase comprises one ion pairing agent. In some embodiments, each of one or more solvents of the mobile phase comprises the same ion pairing agent. In some embodiments, each of one or more solvents of the mobile phase comprises a salt selected from the group consisting of a trietheylammonium salt, tributylammonium salt, hexylammonium salt, dibutylammonium salt, tetrapropylammonium salt, dodecyltrimethylammonium salt, tetra(decyl)ammonium salt, dihexylammonium salt, dipropylammonium salt, myristyltrimethylammonium salt, tetraethylammonium salt, tetraheptylammonium salt, tetrahexylammonium salt, tetrakis(decyl)ammonium salt, tetramethylammonium salt, tetraoctylammonium salt, and tetrapentylammonium salt. In some embodiments, each of one or more solvents of the mobile phase comprises HAA, TBAP, TPAB, TBAC, DBAA, TEAA, DTMAC, TDAB, DHAA, DPAA MTEAB, TEAB, THepAB, THexAB, TrDAB, TMAB, TOAB, TPeABHAA, TBAP, TPAB, TBAC, DBAA, TEAA, DTMAC, or TDAB. A salt of a cation, as used herein, refers to a composition comprising the cation and an anionic counter ion. For example, a “tetrabutylammonium salt” may refer to tetrabutylammonium phosphate, tetrabutylammonium chloride, tetrabutylammonium bromide, tetrabutylammonium phosphate, or another composition comprising the cation tetrabutylammonium and an anionic counter ion. In some embodiments, the ion pairing agent comprises a cation and an anionic counter ion, wherein the cation is selected from the group consisting of trietheylammonium, tributylammonium, hexylammonium, dibutylammonium, tetrapropylammonium, dodecyltrimethylammonium, tetra(decyl)ammonium, dihexylammonium, dipropylammonium, myristyltrimethylammonium, tetraethylammonium, tetraheptylammonium, tetrahexylammonium, tetrakis(decyl)ammonium, tetramethylammonium, tetraoctylammonium, and tetrapentylammonium, and the anionic counter ion is selected from the group consisting of a bromide, chloride, phosphate, and acetate.

Protonated and quaternary amine ion pairing agents can be represented by the following formula:

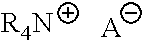

wherein each R independently is hydrogen, optionally substituted aliphatic, optionally substituted heteroaliphatic, optionally substituted aryl or optionally substituted heteroaryl; provided that at least one instance of R is not hydrogen; and A is an anionic counter ion.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups. The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C6-14 aryl”). The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). Suitable anionic counter ions include, but are not limited to, acetate, trifluoroacetate, phosphate, chloride, bromide hexafluorophosphate, sulfate, methylsulfonate, trifluoromethylsulfonate, 1,1,1,3,3,3-hexafluoro-2-propanol (HFIP), 1,1,1,3,3,3-hexafluoro-2-methyl-2-propanol (HFMIP) and the like.

The term “optionally substituted” refers to being substituted or unsubstituted. In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction.

In some embodiments, a solvent solution of the mobile phase (e.g., a first solvent solution or a second solvent solution) comprising at least two ion pairing agents are in a molar ratio of between about 1:1,000 to about 1,000:1, such that the nucleic acids and if present, lipids, traverse the column at different rates. In some embodiments, the at least two ion pairing agents are in a molar ratio between about 1:1,000 to about 1,000:1, 1:900 to about 900:1, 1:800 to about 800:1, 1:700 to about 700:1, 1:600 to about 600:1, 1:500 to about 500:1, 1:400 to about 400:1, about 1:300 to about 300:1, about 1:200 to about 200:1, about 1:100 to about 100:1, about 50:1 to about 1:50, about 40:1 to about 1:40, about 30:1 to about 1:30, about 20:1 to about 1:20, or about 10:1 to about 1:10. In some embodiments, each solvent solution comprises at least two ion pairing agents in a molar ratio of between about 1:100 to about 100:1. In some embodiments, the at least two ion pairing agents are in a molar ratio between about 1:100 to about 100:1, 1:90 to about 90:1, 1:80 to about 80:1, 1:70 to about 70:1, 1:60 to about 60:1, 1:50 to about 50:1, 1:40 to about 40:1, about 1:30 to about 30:1, about 1:20 to about 20:1, about 1:10 to about 10:1, about 5:1 to about 1:5, about 4:1 to about 1:4, about 3:1 to about 1:3, or about 2:1 to about 1:2. In some embodiments, the at least two ion pairing agents are in a 1:1 molar ratio.

In some embodiments, a solvent solution of the mobile phase (e.g., a first solvent solution or a second solvent solution) comprises at least two ion pairing agents that are in a molar ratio of between about 1:6 to about 6:1, such that the nucleic acids and if present, lipids, traverse the column at different rates. In some embodiments, each solvent solution comprises at least two ion pairing agents in a molar ratio of between about 1:4 to about 4:1. In some embodiments, the at least two ion pairing agents are in a molar ratio between about 1:3 to about 3:1, about 1:2 to about 2:1, or about 1:1.5 to about 1.5:1. In some embodiments, the at least two ion pairing agents are in a 1:1 molar ratio.

The concentration of each ion pairing agent in a solvent solution (e.g., a first solvent solution or a second solvent solution) may range from about 1 mM to about 25 M (e.g., about 1 mM, about 2 mM, about 5 mM, about 10 mM, about 50 mM, about 100 mM, about 200 mM, about 500 mM, about 1 M, about 1.2 M, about 1.5 M, about 1.75 M, about 2M, about 2.25 M, about 2.5 M, about 2.75 M, about 3 M, about 3.25 M, about 3.5 M, about 3.75 M, about 4 M, about 4.25 M, about 4.5 M, about 4.75 M, about 5 M, about 5.5 M, about 6 M, about 6.5 M, about 7 M, about 7.5 M, about 8 M, about 8.5 M, about 9 M, about 9.5 M, about 10 M, about 11 M, about 12 M, about 13 M, about 14 M, about 15 M, about 16 M, about 17 M, about 18 M, about 19 M, or about 20 M), inclusive. In some embodiments, the concentration of an ion pairing agent in a mobile phase (e.g., a first solvent solution or a second solvent solution) ranges from about, 10 mM-20 M, 20 mM-15 M, 30 mM-12 M, 40 mM-10 M, 50 mM-8 M, 75 mM-5 M, 100 mM-2.5 M, 125 mM-2 M, 150 mM-1.5 M, 175 mM-1 M, or 200 mM-500 mM. In some embodiments, the concentration of each of the ion pairing agents independently ranges from about, 10 mM-20 M, 20 mM-15 M, 30 mM-12 M, 40 mM-10 M, 50 mM-8 M, 75 mM-5 M, 100 mM-2.5 M, 125 mM-2 M, 150 mM-1.5 M, 175 mM-1 M, or 200 mM-500 mM. In some embodiments, a first or second solvent solution comprises a single ion pairing agent, which is present in an amount from about, 10 mM-20 M, 20 mM-15 M, 30 mM-12 M, 40 mM-10 M, 50 mM-8 M, 75 mM-5 M, 100 mM-2.5 M, 125 mM-2 M, 150 mM-1.5 M, 175 mM-1 M, or 200 mM-500 mM.

The concentration of each ion pairing agent in a solvent solution (e.g., a first solvent solution or a second solvent solution) may range from about 1 mM to about 2 M (e.g., about 1 mM, about 2 mM, about 5 mM, about 10 mM, about 50 mM, about 100 mM, about 200 mM, about 500 mM, about 1 M, about 1.2 M, about 1.5 M, or about 2M), inclusive. In some embodiments, the concentration of an ion pairing agent in a mobile phase (e.g., a first solvent solution or a second solvent solution) ranges from about, 10 mM-1M, 40 mM-300 mM, 50 mM-500 mM, 75 mM-400 mM, 100 mM-300 mM, 200-300 mM, 200-250 mM, or 250-300 mM. In some embodiments, the concentration of each of the ion pairing agents independently ranges from about, 10 mM-1M, 40 mM-300 mM, 50 mM-500 mM, 75 mM-400 mM, 100 mM-300 mM, 200-300 mM, 200-250 mM, or 250-300 mM. In some embodiments, two ion pairing agents are present at concentrations of about 20 mM: 40 mM, 50 mM: 50 mM, 50 mM: 60 mM, 50 mM: 75 mM, 50 mM: 100 mM, 50 mM: 150 mM, 100 mM: 100 mM, 100 mM: 125 mM, 100 mM: 150 mM, 100 mM: 175 mM, 100 mM: 200 mM, 100 mM: 200 mM, 100 mM: 250 mM, 100 mM: 300 mM, 125 mM: 125 mM, 125 mM: 150 mM, 125 mM: 175 mM, 125 mM: 200 mM, 125 mM: 250 mM, 125 mM: 300 mM, 150 mM: 175 mM, 150 mM: 200 mM, 150 mM: 250 mM, 150 mM: 300 mM, 200 mM: 200 mM, 200 mM: 250 mM, 200 mM: 300 mM, 250 mM: 250 mM, 250 mM: 300 mM, or 300 mM: 300 mM.

Examples of ion pairing agent concentrations include but are not limited to 40 mM TEAA: 20 mM DBAA, 100 mM TEAA: 50 mM DBAA, 50 mM TBAP: 50 mM TEAA, 250 mM TBAP: 250 mM TEAA, 300 mM TBAP: 300 mM TEAA, 50 mM TBAP: 150 mM TEAA, 125 mM TBAP: 250 mM TEAA, 250 mM TBAP: 250 mM TEAA, 300 mM TBAP: 300 mM TEAA, 50 mM DBAA: 50 mM TEAA, 60 mM DBAA: 50 mM TEAA, 75 mM DBAA: 50 mM TEAA, 175 mM DBAA: 125 mM TEAA, 100 mM DBAA: 100 mM TEAA, 50 mM TBAP: 100 mM TEAA, 100 mM TBAP: 200 mM TEAA, 125 mM TBAP: 250 mM TEAA, 150 mM TABP: 200 mM TEAA, 150 mM TBAP: 200 mM TEAA, 150 mM TBAP: 250 mM TEAA, 50 mM TBAP: 150 mM TEAA, 100 mM TBAP: 150 mM TEAA, 250 mM TBAP: 200 mM TEAA, 250 mM TBAP: 250 mM TEAA, or 200 mM TBAP: 300 mM TEAA. In some embodiments, one or more solvent solutions of the mobile phase comprise a combination of TPAB and TBAC. In some embodiments, the concentrations of TPAB and TBAC independently range from 50 mM-300 mM. In some embodiments, one or more solvent solutions of the mobile phase comprise 200 mM TPAB: 200 mM TBAC, 250 mM TPAB: 250 mM TBAC, or 300 mM TPAB: 300 mM TBAC. In some embodiments, one or more solvent solutions of the mobile phase comprise 250 mM TPAB: 250 mM TBAC.

Ion pairing agents are generally dispersed within a mobile phase. In HPLC, a mobile phase is an aqueous solution comprising water and/or one or more organic solvents used to carry an HPLC analyte (or analytes), such as a nucleic acid (e.g., mRNA), or mixture of nucleic acids (e.g., mRNAs) through an HPLC column. In some embodiments, a mobile phase for use in HPLC comprises multiple (e.g., 2, 3, 4, 5, or more) solvent solutions. In some embodiments of the HPLC methods, the mobile phase comprises two solvent solutions, a first solvent solution and a second solvent solution (e.g., Mobile Phase A, and Mobile Phase B). In some embodiments, a solvent solution comprises at least two ion pairing agents in a molar ratio of 1:1,000 to 1,000:1. In some embodiments, each solvent solution (e.g., the first solvent solution and the second solvent solution) comprises at least two ion pairing agents in a molar ratio of 1:1,000 to 1,000:1. In some embodiments, a solvent solution comprises at least two ion pairing agents in a molar ratio of 1:100 to 100:1. In some embodiments, each solvent solution (e.g., the first solvent solution and the second solvent solution) comprises at least two ion pairing agents in a molar ratio of 1:100 to 100:1. In some embodiments, a solvent solution comprises at least two ion pairing agents in a molar ratio of 1:75 to 75:1. In some embodiments, each solvent solution (e.g., the first solvent solution and the second solvent solution) comprises at least two ion pairing agents in a molar ratio of 1:75 to 75:1. In some embodiments, a solvent solution comprises at least two ion pairing agents in a molar ratio of 1:50 to 50:1. In some embodiments, each solvent solution (e.g., the first solvent solution and the second solvent solution) comprises at least two ion pairing agents in a molar ratio of 1:50 to 50:1. In some embodiments, a solvent solution comprises at least two ion pairing agents in a molar ratio of 1:25 to 25:1. In some embodiments, each solvent solution (e.g., the first solvent solution and the second solvent solution) comprises at least two ion pairing agents in a molar ratio of 1:25 to 25:1. In some embodiments, a solvent solution comprises at least two ion pairing agents in a molar ratio of 1:10 to 10:1. In some embodiments, each solvent solution (e.g., the first solvent solution and the second solvent solution) comprises at least two ion pairing agents in a molar ratio of 1:10 to 10:1. In some embodiments, a solvent solution comprises at least two ion pairing agents in a molar ratio of 1:6 to 6:1. In some embodiments, each solvent solution (e.g., the first solvent solution and the second solvent solution) comprises at least two ion pairing agents in a molar ratio of 1:6 to 6:1. In some embodiments, a solvent solution comprises at least two ion pairing agents in a molar ratio of 1:4 to 4:1. In some embodiments, each solvent solution (e.g., the first solvent solution and the second solvent solution) comprises at least two ion pairing agents in a molar ratio of 1:4 to 4:1.

In some embodiments, at least one solvent solution of the mobile phase comprises an organic solvent. Generally, an IP-RP HPLC mobile phase comprises a polar organic solvent. Examples of polar organic solvents suitable for inclusion in a mobile phase include but are not limited to alcohols, ketones, nitrates, esters, amides and alkylsulfoxides. In some embodiments, the mobile phase (e.g., at least one solvent solution of the mobile phase) comprises one or more organic solvents selected from the group consisting of polar aprotic solvents, C1-4 alkanols, C1-6 alkanediols, and C2-4 alkanoic acids. In some embodiments, the mobile phase (e.g., at least one solvent solution of the mobile phase) comprises one or more organic solvents selected form the group consisting of acetone, acetonitrile, dimethylformamide, dimethylsulfoxide (DMSO), ethanol, hexylene glycol, isopropanol, methanol, methyl acetate, propanol, and tetrahydrofuran. In some embodiments, the mobile phase (e.g., at least one solvent solution of the mobile phase) comprises acetonitrile. In some embodiments, a mobile phase (e.g., at least one solvent solution of the mobile phase) comprises additional components, for example as described in U.S. Patent Publication US 2005/0011836, which is incorporated herein by reference for this purpose.

The concentration of organic solvent in a mobile phase (e.g., each solvent solution of the mobile phase) can vary. For example, in some embodiments, the volume percentage (v/v) of an organic solvent in a mobile phase varies from 0% (absent) to about 100% of a mobile phase. In some embodiments, the volume percentage of organic solvent in a mobile phase (e.g., at least one solvent solution of the mobile phase) is between about 5% and about 75% v/v. In some embodiments, the volume percentage of organic solvent in a mobile phase (e.g., at least one solvent solution of the mobile phase) is between about 25% and about 60% v/v. In some embodiments, the volume percentage of organic solvent in a mobile phase (e.g., at least one solvent solution of the mobile phase) is at least about 50% v/v. In some embodiments, the volume percentage of organic solvent in a mobile phase (e.g., at least one solvent solution of the mobile phase) is about 50% to about 95%, about 55% to about 90%, about 60% to about 85%, about 65% to about 80%, or about 70% v/v to about 75% v/v. In some embodiments, the concentration of organic solvent in a mobile phase (e.g., at least one solvent solution of the mobile phase) is about 5%, about 10%, about 15%, about 20%, about 25%, about 30%, about 35%, about 40%, about 45%, about 50%, about 55%, about 60%, about 65%, about 70%, about 75%, about 80%, about 85%, about 90% v/v, or about 95% v/v.

In some embodiments, the first solvent solution does not comprise an organic solvent. In some embodiments, the volume percentage of organic solvent in the second solvent solution is at least about 50% v/v. In some embodiments, the volume percentage of organic solvent in the second solvent solution is about 50% to about 95%, about 55% to about 90%, about 60% to about 85%, about 65% to about 80%, or about 70% v/v to about 75% v/v. In some embodiments, the volume percentage of organic solvent in the second solvent solution is about 50%, about 55%, about 60%, about 65%, about 70%, about 75%, about 80%, about 85%, about 90% v/v, or about 95% v/v.

The pH of the mobile phase (e.g., the pH of each solvent solution of the mobile phase) can vary. In some embodiments, the pH of the mobile phase is between about pH 5.0 and pH 9.5 (e.g., about 5.0, about 5.5, about 6.0, about 6.5, about 7.0, about 7.5, about 8.0, about 8.5, about 9.0, or about 9.5). In some embodiments, the pH of the mobile phase is between about pH 6.8 and pH 9.0 (e.g., about 6.8, about 7.0, about 7.2, about 7.4, about 7.6, about 7.8, about 8.0, about 8.3, about 8.5, or about 9.0). In some embodiments, the pH of the mobile phase is about 8.0.

In some embodiments, the pH of the first solvent solution is between about pH 5.0 and pH 9.5 (e.g., about 5.0, about 5.5, about 6.0, about 6.5, about 7.0, about 7.5, about 8.0, about 8.5, about 9.0, or about 9.5). In some embodiments, the pH of the first solvent solution is between about pH 6.8 and pH 9.0 (e.g., about 6.8, about 7.0, about 7.2, about 7.4, about 7.6, about 7.8, about 8.0, about 8.3, about 8.5, or about 9.0). In some embodiments, the pH of the first solvent solution is about 8.0.

In some embodiments, the pH of the second solvent solution is between about pH 5.0 and pH 9.5 (e.g., about 5.0, about 5.5, about 6.0, about 6.5, about 7.0, about 7.5, about 8.0, about 8.5, about 9.0, or about 9.5). In some embodiments, the pH of the second solvent solution is between about pH 6.8 and pH 9.0 (e.g., about 6.8, about 7.0, about 7.2, about 7.4, about 7.6, about 7.8, about 8.0, about 8.3, or about 8.5). In some embodiments, the pH of the second solvent solution is about 8.0.

The concentration of two or more solvent solutions in a mobile phase can vary. For example, in a mobile phase comprising two solvent solutions (e.g., a first solvent solution and a second solvent solution), the volume percentage of the first solvent solution may range from about 0% (absent) to about 100%. In some embodiments, the volume percentage of the first solvent solution may range from about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% v/v.

Conversely, in some embodiments, the volume percentage of the second solvent solution of a mobile phase may range from about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% v/v.

In some embodiments, the ratio of the first solvent solution to the second solvent solution is held constant (e.g., isocratic) during elution of the nucleic acid. However, the skilled artisan will appreciate that in other embodiments, the relative ratio of the first solvent solution to the second solvent solution can vary throughout the elution step. For example, in some embodiments, the ratio of the first solvent solution is increased relative to the second solvent solution during the elution step. In some embodiments, the ratio of the first solvent solution is decreased relative to the second solvent solution during the elution step.

The concentration of one or more ion pairing agents in a mobile phase (e.g., a solvent solution) can vary. The relative ratios of the at least two ion pairing agents in a mobile phase (or solvent solution) may vary or be held constant (e.g., isocratic) during the eluting step. In some embodiments, the ratio of a first ion pairing agent is increased relative to a second ion pairing agent during the elution step. In some embodiments, the ratio of a first ion pairing agent is increased relative to a second ion pairing agent during the elution step. For example, in some embodiments, the ratio of TPAB to TBAC ranges from about 4:1 to about 1:4, about 3:1 to about 1:3, about 2:1 to about 1:2, or about 1:1 to 1:3.

The mobile phase (e.g., a solvent solution) may be gradient or isocratic with respect to the concentration of one or more organic solvents.

The pH of the mobile phase can vary. In some embodiments, the pH of the mobile phase is between about pH 5.0 and pH 9.5 (e.g., about 5.0, about 5.5, about 6.0, about 6.5, about 7.0, about 7.5, about 8.0, about 8.5, about 9.0, or about 9.5). In some embodiments, the pH of the mobile phase is between about pH 6.8 and pH 8.5 (e.g., about 6.8, about 7.0, about 7.2, about 7.4, about 7.6, about 7.8, about 8.0, about 8.3, or about 8.5). In some embodiments, the pH of the mobile phase is about 7.0.

### Nucleic Acids

Some aspects relate to methods of producing nucleic acids, including messenger ribonucleic acid (mRNA). Messenger RNA (mRNA) is RNA that encodes a (at least one) protein or a fragment thereof and can be translated to produce the encoded protein or fragment in vitro, in vivo, in situ, or ex vivo. mRNA comprises an open reading frame (ORF) encoding the protein or fragment thereof. In some embodiments, the mRNA further comprises a 5′ untranslated region (UTR), 3′ UTR, a polyA tail, and/or a 5′ cap analog.

The mRNA may encode a single protein or fragment or it may be a polycistronic mRNA, which encodes more than one protein or fragment separately within the same mRNA molecule. Additionally or alternatively, the mRNA may encode a fusion protein or fragment thereof.

i. Open Reading Frame (ORF)

An open reading frame (ORF) is a continuous stretch of DNA or RNA beginning with a start codon (e.g., methionine (ATG or AUG)) and ending with a stop codon or codons (e.g., TAA, TAG, TGA, UAA, UAG, UGA, UGAUGA or UGAUAAUAG). For clarity: the stop codon itself is not considered a part of the ORF. An ORF typically encodes a protein or fragment thereof.

ii. Untranslated Regions (UTRs)

In some embodiments, mRNA comprises one or more regions or parts which act or function as an untranslated region. A 5′ untranslated region” (5′ UTR) is a region of an mRNA that is upstream (i.e., 5′) from the start codon and does not encode a polypeptide. A 3′ untranslated region” (3′UTR) is a region of an mRNA that is downstream (i.e., 3′) from the stop codon and also does not encode a polypeptide.

The 5′ UTR may start at the transcription start site and continues to the start codon but does not include the start codon. The 3′ UTR may start immediately following the stop codon and continue until a transcriptional termination signal. A variety of 5′ UTR and 3′ UTR sequences are known. Exemplary UTR sequences include SEQ ID NOs: 1, 2, and 5-36 (5′ UTRs) and 3, 4, and 37-44 (3′ UTRs), which are shown in Tables S-1 (5′ UTRs) and S-2 (3′ UTRs) of the section “Exemplary Sequences”. In some embodiments, the 5′ UTR comprises a sequence provided in Table S-1 or a sequence with at least 80%, 85%, 90%, 95%, 96%, 97%, 98%, 99% or 100% identity to a 5′ UTR sequence provided in Table S-1, or a variant or a fragment thereof. In some embodiments, the 3′ UTR comprises a sequence provided in Table S-2 or a sequence with at least 80%, 85%, 90%, 95%, 96%, 97%, 98%, 99% or 100% identity to a 3′ UTR sequence provided in Table S-2, or a variant or a fragment thereof.

Each RNA species in a multivalent RNA composition may comprise an IDR sequence that is not a sequence isomer of an IDR sequence of another RNA species in a multivalent RNA composition (e.g., the IDR sequence does not have the same number of adenosine nucleotides, the same number of cytosine nucleotides, the same number of guanine nucleotides, and the same number of uracil nucleotides (and consequently the same mass) as another IDR sequence in the composition, even if those sequences have different sequences).

Each RNA species in a multivalent RNA composition may comprise an IDR sequence having a mass that differs from the mass of IDR sequences of each other RNA species in a multivalent RNA composition. For example, the mass of each IDR sequence may differ from the mass of other IDR sequences by at least 9 Da, at least 25 Da, at least 25 Da, or at least 50 Da. Use of IDR sequences with distinct masses allows RNA fragments comprising different IDR sequences to be distinguished using mass-based analysis methods (e.g., mass spectrometry), which do not require reverse transcription, amplification, or sequencing of RNAs.

Each RNA species in an RNA composition may comprises an IDR sequence with a different length. For example, each IDR sequence may have a length independently selected from 0 to 25 nucleotides. The length of a nucleic acid influences the rate at which the nucleic acid traverses a chromatography column, and so the use of IDR sequences of different lengths on different RNA species allows RNA fragments having different IDR sequences to be distinguished using chromatography-based methods (e.g., LC-UV).

Combinations of features may be included in flanking regions and may be contained within other features. For example, the ORF may be flanked by a 5′ UTR which may contain a strong Kozak translational initiation signal and/or a 3′ UTR which may include an oligo (dT) sequence for templated addition of a poly-A tail. 5′ UTR may comprise a first polynucleotide fragment and a second polynucleotide fragment from the same and/or different genes such as the 5′ UTRs described in US 2010/0293625 and WO 2015/085318.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US 2010/0129877.

For the purposes of the present disclosure, a UTR may also include one or more translation enhancer elements (TEE). As a non-limiting example, the TEE may include those described in US 2009/0226470, herein incorporated by reference, and those known in the art.

iii. PolyA Tail

In some embodiments, the mRNA contains a 3′-polyA tail. A polyA tail may contain 10 to 300 adenosine monophosphates. It can, in some instances, comprise up to about 400 adenine nucleotides. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine nucleotides. In some embodiments, a polyA tail contains 50 to 250 adenosine nucleotides. In some embodiments, a polyA tail has a length of about 50, about 100, about 150, about 200, about 250, about 300, about 350, or about 400 nucleotides. In some embodiments, a polyA tail has a length of 100 nucleotides.

In some embodiments, an mRNA may comprise two polyA sequences separated by an intervening nucleotide sequence. In some embodiments, the intervening nucleotide sequence comprises no more than 3, no more than two, no more than 1, or no adenosine nucleotides. In some embodiments, the intervening sequence comprises 3 adenosine nucleotides. In some embodiments, the intervening sequence is no more than 30, no more than 25, no more than 20, no more than 15, or no more than 10 nucleotides long. In some embodiments, the intervening sequence consists of 10 nucleotides. In some embodiments, the intervening sequence comprises the sequence of GCAUAUGACU (SEQ ID NO: 50). In some embodiments, the intervening sequence does not begin with an adenosine nucleotide, and does not end with an adenosine nucleotide. In some embodiments, the first polyA sequences comprises at least 15, at least 20, at least 25, or at least 30 consecutive adenosine nucleotides. In some embodiments, the second polyA sequences comprises at least 55, at least 60, at least 65, or at least 70 consecutive adenosine nucleotides. In some embodiments, the first polyA sequence comprises 30 consecutive adenosine nucleotides. In some embodiments, the second polyA sequence comprises 70 adenosine nucleotides.

### iv. 5′ Cap

In some embodiments, mRNA comprises a 5′ end cap or a “5′ terminal cap.” A cap analog may be, for example, a dinucleotide cap, a trinucleotide cap, or a tetranucleotide cap. In some embodiments, a cap analog is a dinucleotide cap. In some embodiments, a cap analog is a trinucleotide cap. In some embodiments, a cap analog is a tetranucleotide cap.

5′-capping of polynucleotides may be completed concomitantly during an in vitro transcription reaction using, for example, the following chemical RNA cap analogs to generate the 5′-guanosine cap structure according to manufacturer protocols: 3′-O-Me-m7G(5′)ppp(5′)G [the ARCA cap];G(5′)ppp(5′) A; G(5′)ppp(5′)G; m7G(5′)ppp(5′) A; m7G(5′)ppp(5′)G (New England BioLabs, Ipswich, MA). 5′-capping of modified mRNA may be completed post-transcriptionally using, for example, a Vaccinia Virus Capping Enzyme to generate the “Cap 0” structure: m7G(5′)ppp(5′)G (New England BioLabs, Ipswich, MA). A Cap 1 structure may be generated using both Vaccinia Virus Capping Enzyme and a 2′-O methyl-transferase to generate: m7G(5′)ppp(5′)G-2′-O-methyl. A Cap 2 structure may be generated from the Cap 1 structure followed by the 2′-O-methylation of the 5′-antepenultimate nucleotide using a 2′-O methyl-transferase. A Cap 3 structure may be generated from the Cap 2 structure followed by the 2′-O-methylation of the 5′-preantepenultimate nucleotide using a 2′-O methyl-transferase. Enzymes may be derived from a recombinant source. Other cap analogs, such as a 7 mG(5′)ppp(5′) NlmpNp cap, may be used.

### Chemical Modifications

An mRNA may include nucleotides that are not chemically modified (i.e., unmodified nucleotides), nucleotides that are chemically modified, or both. Nucleotides that are not chemically modified are the standard ribonucleotides having adenosine, guanosine, cytidine, or uridine nucleosides.

Some embodiments of mRNAs comprise modified nucleosides and/or nucleotides. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside in combination with a phosphate group. Modifications to nucleotides or nucleosides can be at the sugar or nucleobase. Modified nucleotides may by synthesized by any useful method, such as, for example, chemically, enzymatically, or recombinantly.

In some embodiments, modified nucleobases in nucleic acids (e.g., RNA, such as mRNA) comprise N1-methyl-pseudouridine (mlψ), N1-ethyl-pseudouridine (elψ), 5-methoxy-uridine (mo5U), 5-methyl-uridine (m5U), 5-methyl-cytidine (m5C), and/or pseudouridine (ψ). In some embodiments, modified nucleobases in nucleic acids (e.g., RNA, such as mRNA) comprise 5-methoxymethyl uridine, 5-methylthio uridine, 1-methoxymethyl pseudouridine, 5-methyl cytidine, and/or 5-methoxy cytidine. In some embodiments, the mRNA includes a combination of at least two (e.g., 2, 3, 4 or more) of any of the aforementioned modified nucleobases, including but not limited to chemical modifications.

In some embodiments, a mRNA comprises 1-methyl-pseudouridine (mlψ) substitutions at one or more or all uridine positions of the mRNA.

In some embodiments, a mRNA comprises 1-methyl-pseudouridine (mlψ) substitutions at one or more or all uridine positions of the nucleic acid and 5-methyl cytidine substitutions at one or more or all cytidine positions of the mRNA.

In some embodiments, a mRNA comprises pseudouridine (ψ) substitutions at one or more or all uridine positions of the mRNA.

In some embodiments, a mRNA comprises pseudouridine (ψ) substitutions at one or more or all uridine positions of the nucleic acid and 5-methylcytidine substitutions at one or more or all cytidine positions of the mRNA.

In some embodiments, a mRNA comprises uridine at one or more or all uridine positions of the mRNA.

In some embodiments, a mRNA comprises 5-methyl-uridine and 5-methyl cytidine at one or more or all uridine and cytidine positions, respectively, of the mRNA.

In some embodiments, mRNAs are uniformly modified (e.g., fully modified, modified throughout the entire sequence) for a particular modification. For example, a mRNA can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above. In some embodiments, the ORF is uniformly modified for a particular modification, such as 1-methyl-pseudouridine. In some embodiments, the uniform modification does not include the mRNA cap. For instance, a cap with different modifications from the remainder of the mRNA can be added co-transcriptionally or post-transcriptionally to the mRNA.

### Codon Optimization

In some embodiments, an ORF encoding a protein or fragment thereof is codon optimized. Codon optimization methods are known in the art. For example, an ORF of any one or more of the sequences listed below may be codon optimized. Codon optimization, in some embodiments, may be used to match codon frequencies in target and host organisms to ensure proper folding; bias GC content to increase RNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and RNA degradation sites; adjust translational rates to allow the various domains of the protein to fold properly; or reduce or eliminate problem secondary structures within the polynucleotide. Codon optimization tools, algorithms and services are known in the art-non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95%, less than 90%, less than 85%, less than 80%, or less than 75% sequence identity to a naturally-occurring or wild-type sequence open reading frame (e.g., a naturally-occurring or wild-type mRNA sequence encoding a protein or fragment thereof). In some embodiments, a codon optimized sequence shares between 65% and 85% sequence identity to a naturally-occurring or wild-type sequence (e.g., a naturally-occurring or wild-type RNA or DNA sequence encoding a protein or fragment thereof).

### EXAMPLES

### Example 1: Monitoring In Vitro Transcription (IVT) Reaction Rates and Endpoints Using Raman Spectroscopy

This example describes the monitoring of in vitro transcription of mRNA using Raman spectroscopy.

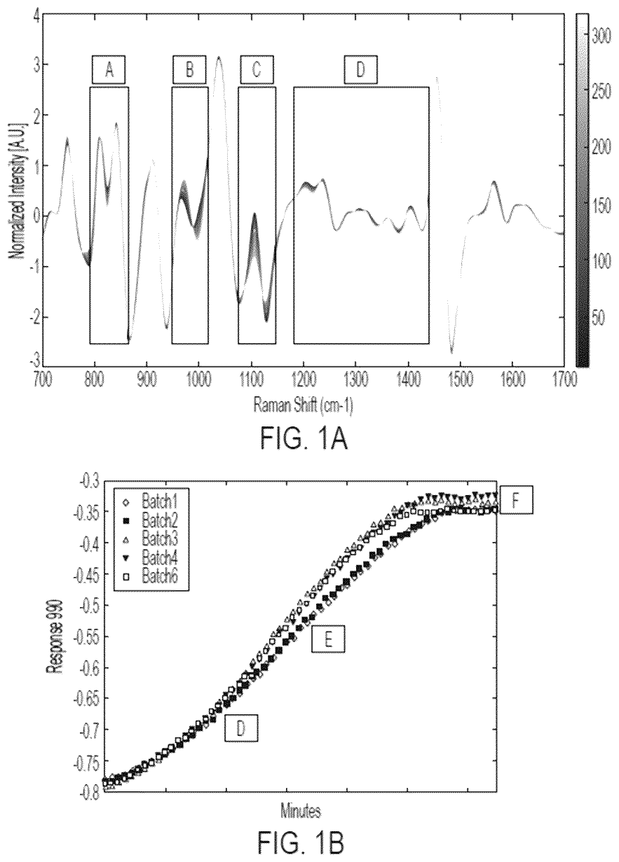

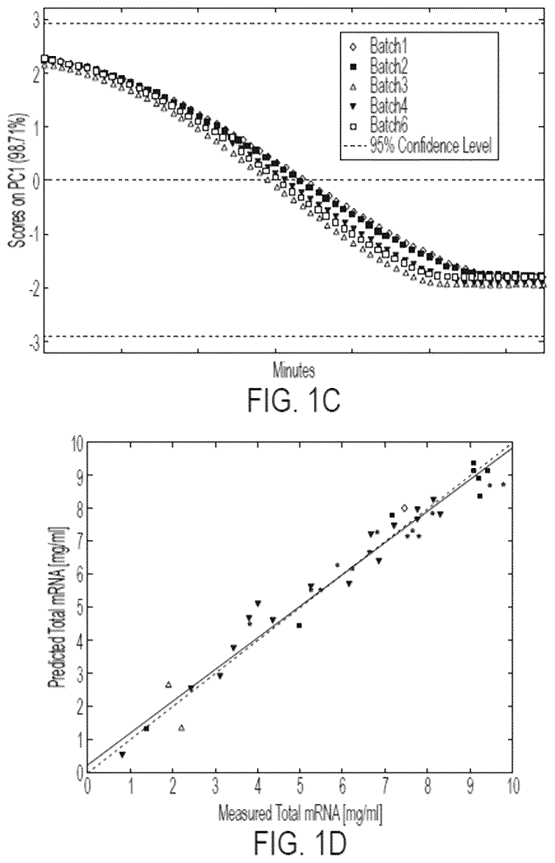

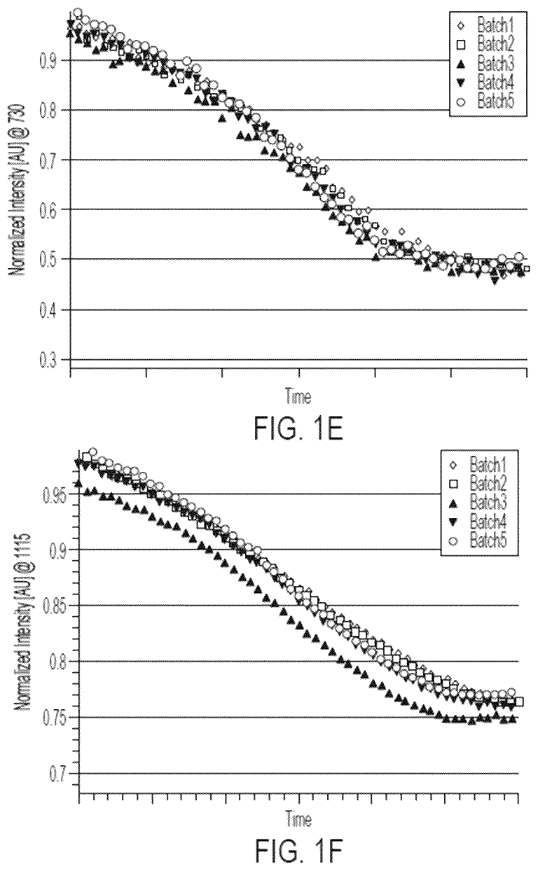

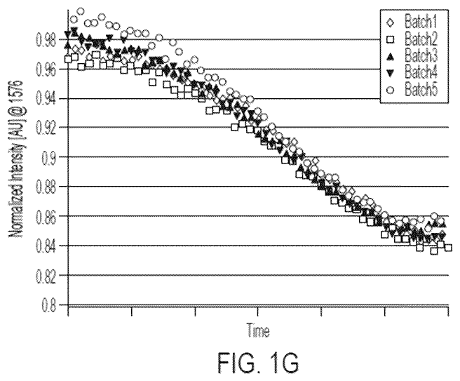

A 100 mL batch of in vitro transcription of mRNA was run at 37° C. using a T7 polymerase enzyme. The reaction was monitored over time using Raman spectroscopy. An overlay of all of the spectra obtained with grayscale to show time (with black being the first spectrum and white being the last spectrum) is presented in FIG. 1A. Each spectrum is an average of multiple spectra surrounding a given timepoint to reduce noise. From FIG. 1A, it was determined which peaks were expected to change over time and how they were expected to change, and which peaks were expected to stay the same over time.

To monitor the rate of in vitro transcription of mRNA, the normalized intensity (y-axis, labeled Response 990) of an orthophosphate peak (representative of total mRNA) was plotted versus time (in minutes), as shown in FIG. 1B. It was determined that the rate of reaction for in vitro mRNA transcription may be determined using Raman spectroscopy. For example, the rate of reaction may be determined from FIG. 1B using the following equation:

$$Rate of Reaction ( R ) = ( {I}_{2} - {I}_{1} ) / ( {T}_{2} - {T}_{1} )$$

as discussed in more detail elsewhere herein. For example, in FIG. 1B, the first timepoint for determining the rate of reaction may be that represented by box D and the second timepoint for determining the rate of reaction may be that represented by box E, as the portion of the curve between box D and box E is linear.

It was further determined that the endpoint could be identified using Raman spectroscopy. For example, as shown in FIG. 1B, the endpoint of the in vitro mRNA transcription was identified as the time when the normalized intensity of the orthophosphate peak (representative of total mRNA) leveled off and no longer continued to increase at a significant rate (see, e.g., the timepoint represented by box F in FIG. 1B).

Similarly, it was further determined that the endpoint could be predicted using Raman spectroscopy. For example, in some embodiments, once the rate of reaction and the intensity of the endpoint (e.g., of the orthophosphate peak, which is representative of total mRNA) are determined as described above, the timing of the endpoint may be determined for future batches with the same yield using the following equation:

$${T}_{end} = ( {I}_{end} ) / R$$

where T_{end} is the time at the endpoint, I_{end} is the normalized intensity of the orthophosphate peak (representative of total mRNA) peaks at the endpoint, and R is the rate of reaction determined as described above.

Moreover, it was determined that, in some embodiments, these determinations (e.g., rate of reaction, endpoint intensity, and/or endpoint time) can be made using PCA (see FIG. 1C) using the full spectrum or using any portions of the spectrum (e.g., portions of the spectrum representative of the concentration of one or more reactants, the concentration of one or more products, and/or the concentration of one or more byproducts) that are directly correlated with the in vitro transcription of mRNA (e.g., they increase or decrease at the same rate relative to the rate of in vitro transcription). When using other portions of the spectrum, the equations above may be modified such that I (e.g., I₂, I₁, and I_{end}) represents the value on the y-axis at that time, and T (e.g., T₂, T₁, and T_{end}) represents the value on the x-axis. When using other portions of the spectrum, the endpoint may still be identified as the point where the y-axis values level off and no longer change significantly.

For example, it was determined that monitoring a Raman peak associated with orthophosphate ([HPO₄]²⁻) (e.g., at 990 cm⁻¹) may be used to monitor in vitro mRNA transcription, in certain embodiments, rather than directly monitoring peaks associated with the mRNA itself. During in vitro transcription, in some embodiments, each NTP addition results in formation of inorganic pyrophosphate (PPi), which is hydrolyzed by pyrophosphatase (PPase) to orthophosphate, as shown in the following equations:

$${( RNA )}_{n} + {MgNTP ( RNA )}_{n + 1} \to {MgP}_{2} {O}_{7}^{2 -} + {H}^{+} \\ {MgP}_{2} {O}_{7}^{2 -} + {H}_{2} O ⇄ 2 {HPO}_{4}^{2 -} + {Mg}^{2 +}$$

Accordingly, determination of the rate of formation (or amount) of orthophosphate allows determination of the rate of formation (or amount) of the mRNA, as 2 moles of orthophosphate are formed for every mole of NTP consumed, in some embodiments.

The calculated concentration of total mRNA based on observing the portion of the Raman spectrum associated with orthophosphate at a given time point was plotted versus the concentration of total mRNA at the same timepoint as measured using HPLC (see FIG. 1D). As shown in FIG. 1D, determining the concentration of total mRNA by observing the portion of the Raman spectrum associated with orthophosphate worked well as plotting the calculated versus measured values had an R² value of approximately 0.96.

It was also determined that monitoring a Raman peak associated with reactants (e.g., total NTPs, or individual NTPs, such as ATP or GTP) may be used to monitor in vitro mRNA transcription, in certain embodiments, as the concentration of reactants will decrease as in vitro mRNA transcription progresses. For example, FIG. 1E shows how ATP levels decreased as in vitro mRNA transcription progressed for five batches, FIG. 1F shows how total NTP levels decreased as in vitro mRNA transcription progressed for five batches, and FIG. 1G shows how GTP levels decreased as in vitro mRNA transcription progressed for five batches.

These findings indicate that Raman spectroscopy is useful for monitoring in vitro transcription of mRNA, including determination of the rate of reaction and endpoint.

### Example 2: Continuous In Vitro Transcription (IVT) Using Adaptive Residence Time Adjustment Informed by Raman Spectroscopy

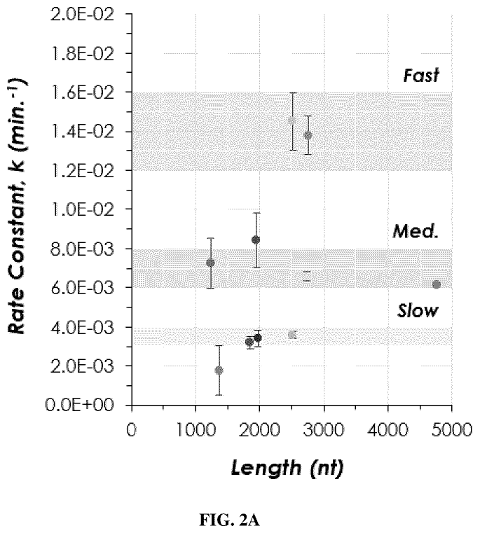

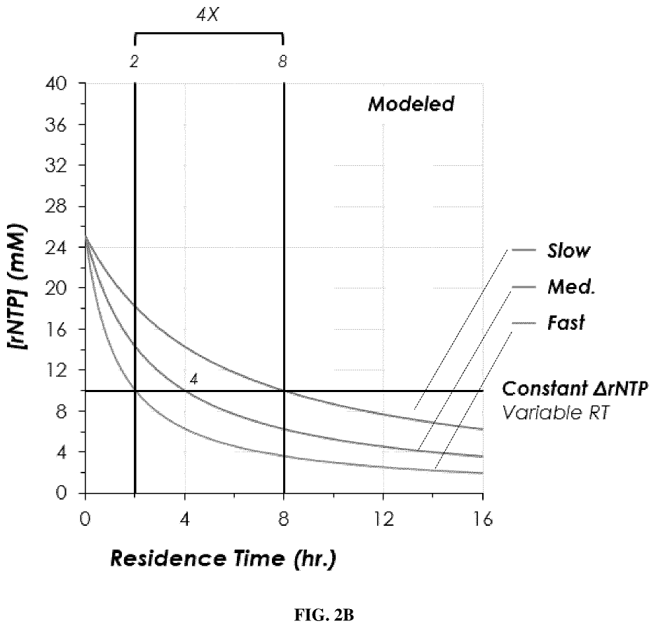

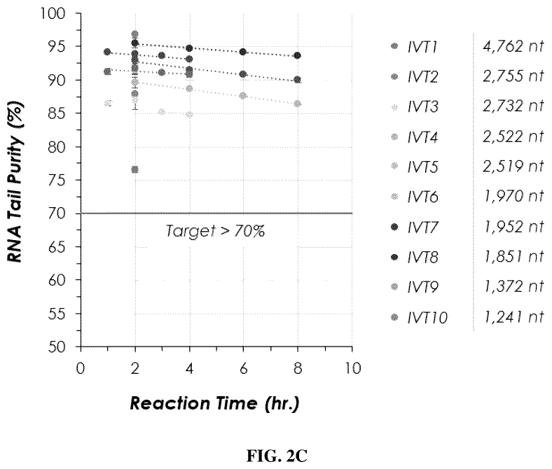

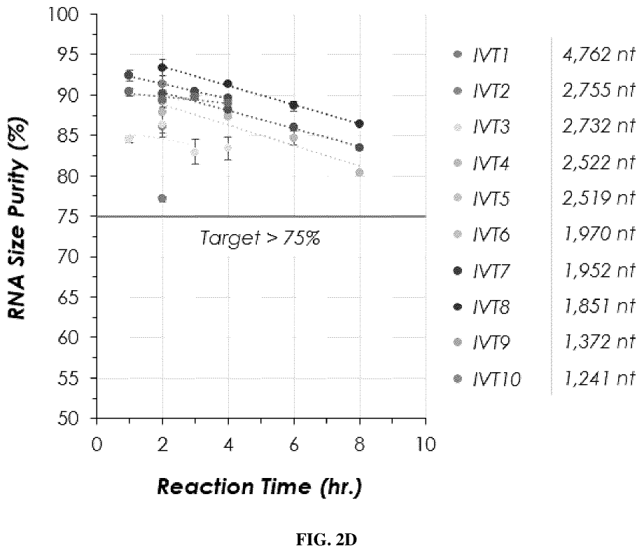

Raman spectroscopy was used to monitor reaction rates and kinetics of several IVT reactions transcribing different mRNA sequences. It was determined that certain sequences, independent of length, exhibited different IVT rate constants (FIG. 2A). Modeling based on these observed reaction rates, binned as “slow”, “medium”, or “fast”, indicated different residence times were warranted to achieve a desired reaction rate, in terms of change in NTP concentration (FIG. 2B). For instance, a sequence with a lower (slow) rate constant would need a 4-fold longer residence time for an IVT reaction to proceed at the same rate as transcription of a (faster) sequence with a higher rate constant. Increasing residence time, however, can reduce RNA purity, in terms of polyA tailing efficiency (FIG. 2C) and production of RNAs with expected size (FIG. 2D).

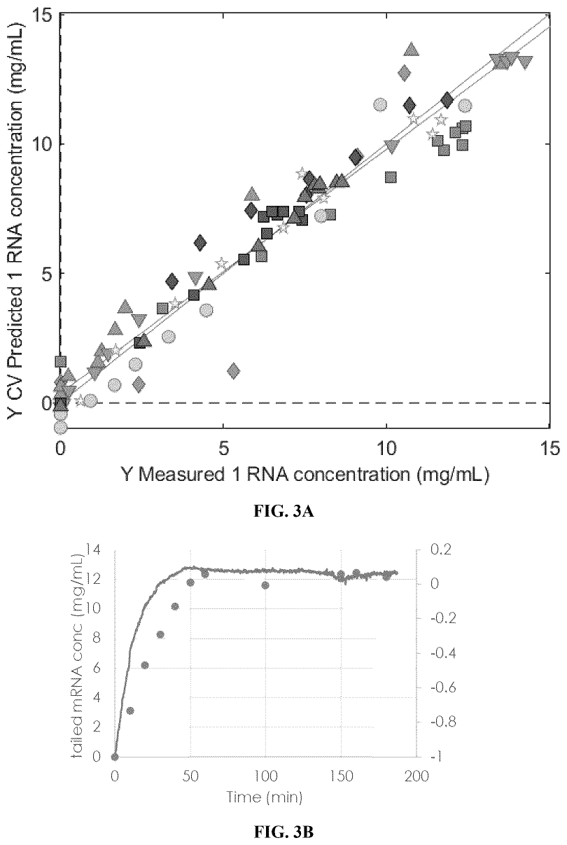

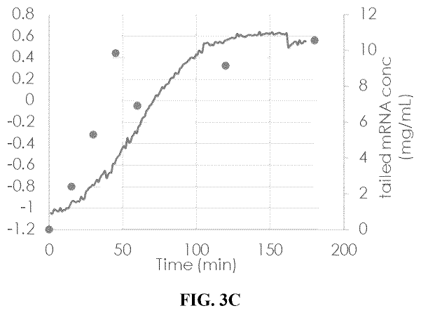

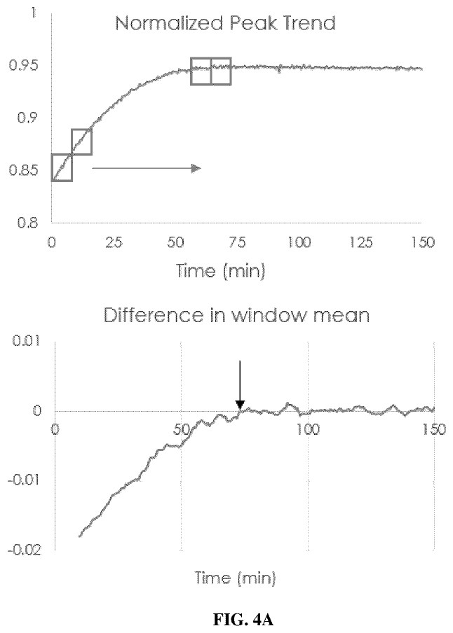

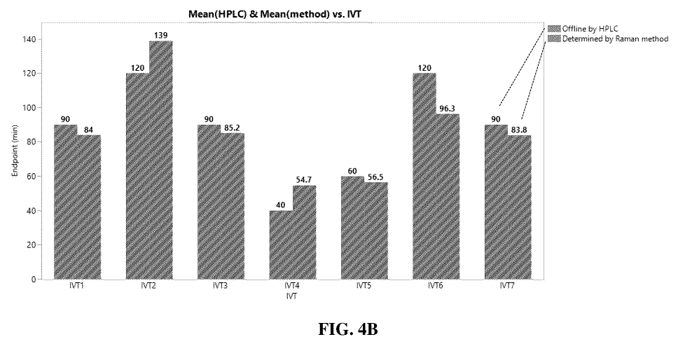

Raman spectroscopy was employed to tailor IVT residence time to the RNA sequence being transcribed, to determine where longer or shorter residence times are warranted, while avoiding the reduced purity and lower yield that can result from unnecessary increases in residence time. Predicted RNA concentrations using Raman spectroscopy correlated well with measured RNA concentrations, and Raman-based predictions were accurate over time for multiple RNA sequences (FIGS. 3A-3C). These kinetic measurements were used to monitor reaction rates and predict reaction endpoints (FIG. 4A), which correlated well with empirically determined endpoints measured by HPLC analysis of mixtures at different times (FIG. 4B).

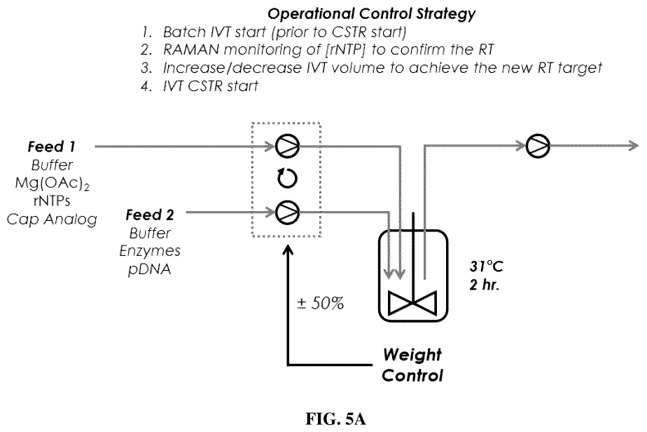

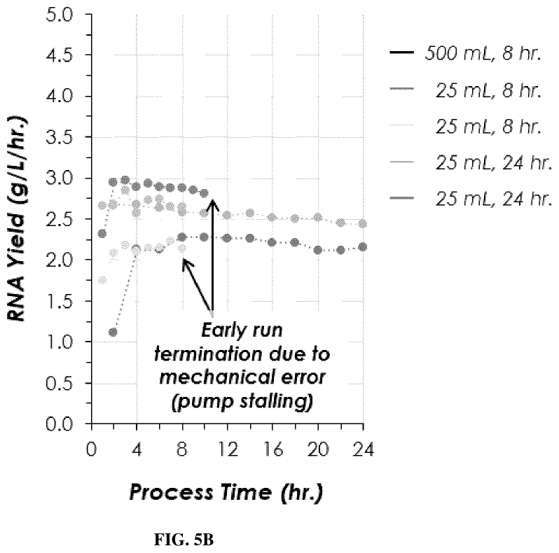

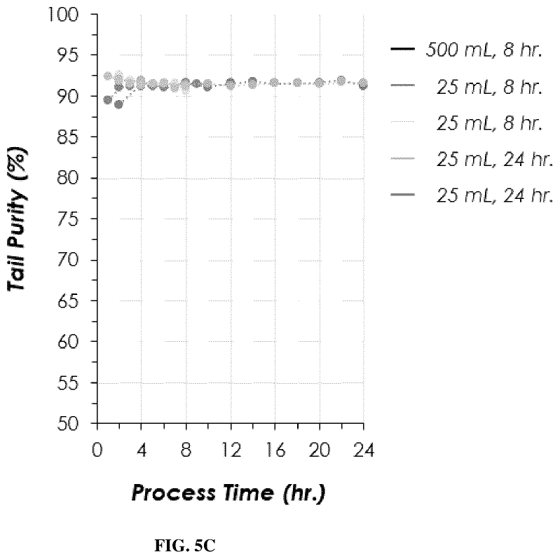

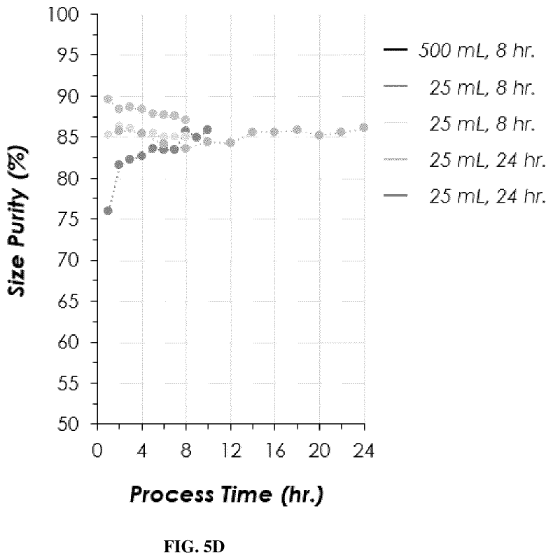

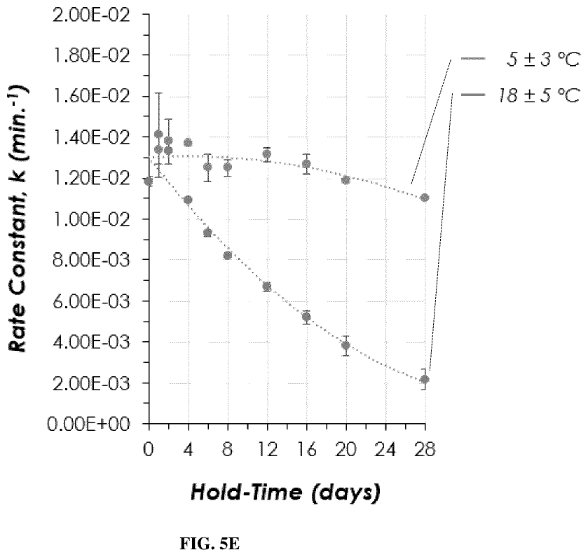

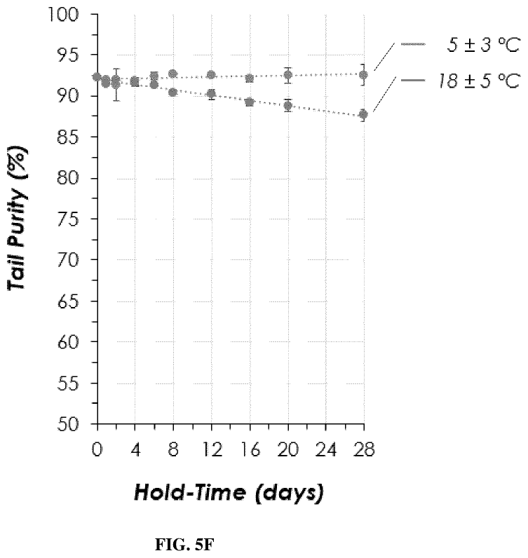

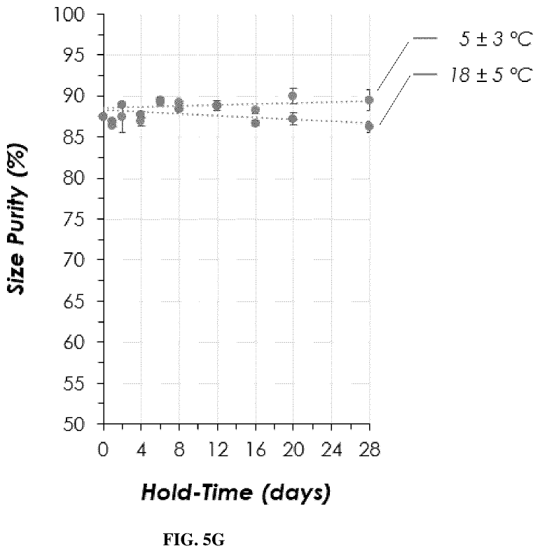

This tailoring of residence time was implemented in a CSTR, as shown in FIG. 5A. Two input feed solutions, one containing Mg(OAc)₂, NTPs, cap analog, and buffer, and another containing buffer, RNA polymerase, and plasmid DNA template, were input into the CSTR, with which a Raman sensor was coupled. As the Raman sensor measured NTP concentration, the change in NTP concentration was used to determine reaction rate, endpoint, and target residence time, with volume being modified to achieve the determined target residence time. Results of this continuous process are shown in FIGS. 5B-5D, indicating that RNA yield, tail purity, and size purity were consistent following the period of measurement and residence time adjustment in the first 4 hours. Feed solutions stored at 5° C. for up to 20 days achieved similar rate constants (FIG. 5E), tail purity (FIG. 5F), and size purity (FIG. 5G), indicating that IVT reagent stability is not a barrier to long-term continuous IVT processes, such as those described in this Example. These results indicate that Raman-based monitoring of IVT reaction conditions can effectively inform adjustment of reaction parameters (e.g., residence time, volume, and reactant input and output) to account for sequence-specific variability in transcription rates, thereby maintaining consistently high RNA yield and purity for a given RNA sequence.

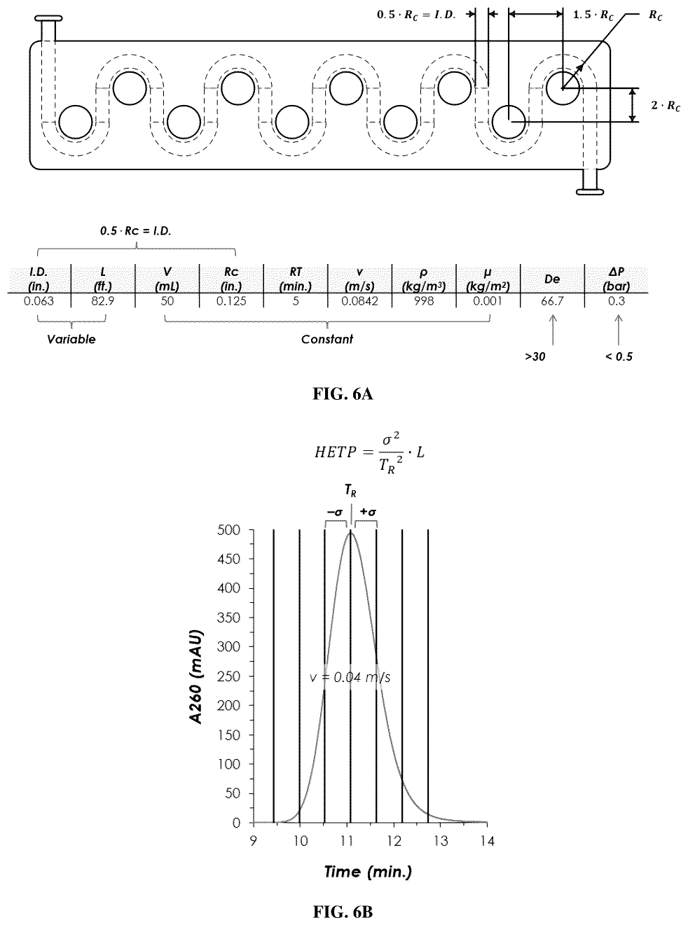

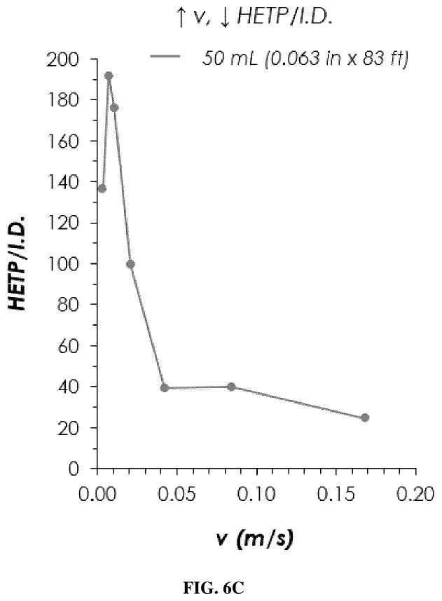

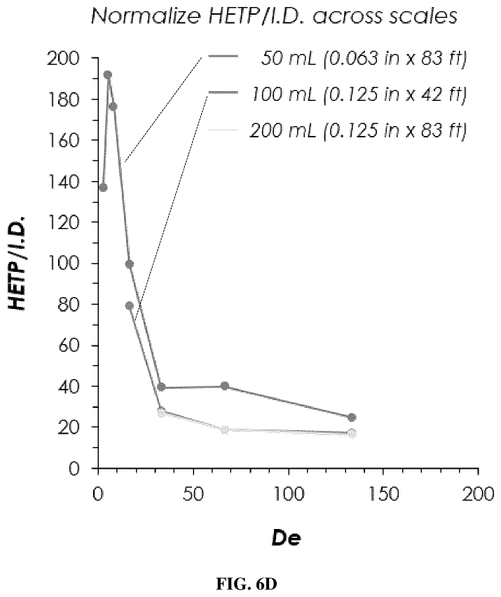

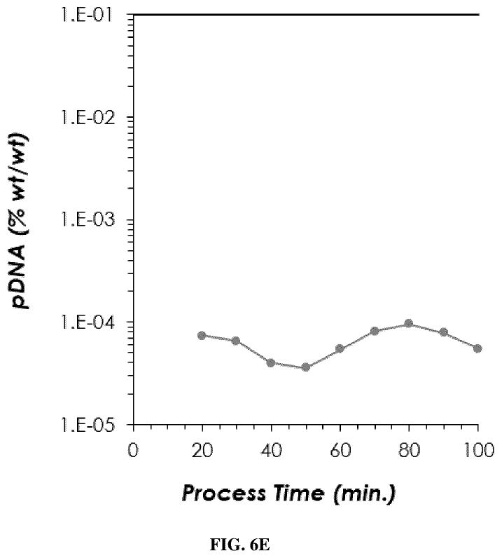

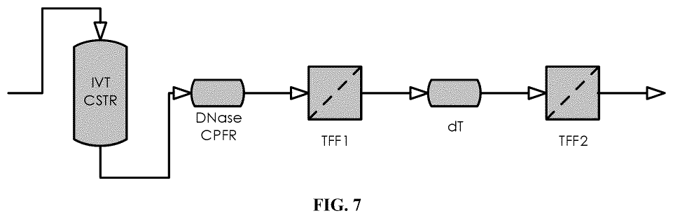

To facilitate continuous DNase digestion in conjunction with continuous IVT, a DNase Dean Vortices continuous plug flow reactor (CPFR) was designed, as shown in FIG. 6A. Such a reactor design allows increased radial diffusion, increased axial dispersion, lower residence time distribution (RTD), reduced pipe length, and reduced pressure drop, which are beneficial for use in digesting DNA in a mixture following completion of IVT to a desired extent. Performance was evaluated using an ATP pulse tracer, with absorbance at 260 nm being measured over time to calculate height equivalent to a theoretical plate (HETP), as shown in FIG. 6B. HETP relative to I.D. was consistent at velocities 0.04 m/s and above (FIG. 6C), or Dean numbers 30 and above (FIG. 6D). Use of this reactor system reduced plasmid DNA contamination (% wt/wt) to levels below 0.0001% wt/wt, 1,000-fold lower than the 0.1% wt/wt threshold target for producing therapeutic or prophylactic mRNA (FIG. 6E). These results indicate that a CPFR with Dean vortices allows effective DNase digestion for removal of DNA templates from in vitro-transcribed RNA compositions. In combination with continuous IVT methods, such CPFR reactors allow continuous production in vitro transcribed RNA compositions with minimal contaminating DNA content.

### Example 3: CSTR System Alterations in Response to Raman Spectroscopy Results

At least one feed solution is delivered to the continuous stir tank reactor (CSTR) at an initial flow rate. Two feed solutions, one containing NTPs and cap (or cap analog), and another containing a DNA template and RNA polymerase, may be delivered from separately to form an IVT reaction mixture in the CSTR. The reaction is monitored over time using Raman spectroscopy. The rate of in vitro transcription and endpoint are determined as described in Example 1.

Where Raman spectroscopy determines that a longer residence time is desired (e.g., if the concentration of unincorporated NTPs present in the IVT reaction mixture being output from the CSTR is higher than desired (e.g., greater than 20% an input concentration of NTPs), the volume of the IVT reaction mixture is increased. Volume may be increased by increasing the inlet flow rate(s) and/or decreasing the outlet flow rate.

The desired volume is calculated by using a CSTR design equation based on the Raman-determined reaction rate, Raman-determined endpoint, and desired endpoint. Continued monitoring of the reaction rate and endpoint determination using Raman spectroscopy then identifies whether the change in the volume is sufficient to change the residence time such that the desired reaction rate and/or endpoint are obtained. The volume may be further increased or increased again to obtain a longer residence time.

Conversely, where Raman spectroscopy and analysis determined that a shorter residence time is desired (e.g., productivity per unit time may be increased without undue loss of reactants, or without increasing the amount of unincorporated NTP loss beyond a desired threshold), the volume of the IVT reaction mixture is reduced. Volume may be reduced by decreasing the inlet flow rate(s).

In either case, if the change in volume results in a residence time that exceeds or undershoots the target residence time, the volume may be further adjusted to achieve a residence time approximating the target residence time (e.g., within 80% to 120% of the target residence time). A control schematic may be implemented to adjust the volume in real-time to account for variations in the reaction rate over time.

### Example 4: PFR System Alterations in Response to Raman Spectroscopy Measurements on Separated Starting Batch

A preliminary IVT reaction is conducted in a separate batch reaction vessel, with Raman spectroscopy monitoring over time to determine the rate of in vitro transcription as described in Example 1. The preliminary IVT reaction is used in order to identify the target residence time for the mRNA sequence being transcribed, so that an IVT reaction for producing that mRNA sequence may be implemented in a larger PFR using that target residence time. Based on this target residence time, an IVT reaction in the PFR system is calibrated such that the PFR length and inlet flow rate allow for the IVT reaction to proceed in the PFR with a residence time at or within a desired range (e.g., 80% to 120%) of the target residence time.

Accordingly, valves along the PFR are open and shut, such that the active portion of the PFR provides a volume suitable to allow the IVT reaction mixture to proceed with the target residence time. Once this is completed the inlet feed is delivered to the PFR system. Once steady state is reached, the outlet stream contains an IVT reaction mixture that has been present in the PFT for approximately (e.g., 80% to 120% of) the target residence time.

### Example 5: PFR System Alterations in Response to Raman Spectroscopy Measurements Throughout the PFR System

Multiple Raman sensors are located periodically along the length of a PFR. The spacing of these sensors may vary, but generally sensors are positioned such that Raman spectra collected at various points to detect changes in NTP concentration between two different collection points, such that the rate of in vitro transcription may be determined.

At least one feed solution is delivered to the PFR system at an initial flow rate. Raman sensors obtain spectra at points along the length of the PFR, which are used to determine the reaction rate, rate constant for RNA sequence being transcribed, and consequently the target residence time suitable for achieving a target endpoint (e.g., NTP concentration below a given threshold) for that RNA sequence. Residence time of the IVT reaction mixture is modified as needed to a time within a desired range (e.g., 80% to 120%) of the target residence time. For example, an outlet valve may be opened after the position first Raman sensor which indicates that a desired endpoint (e.g., threshold consumption of NTPs) has been reached, or the active length of the PFR may be extended if a longer residence time is warranted.

Residence time may be modified by opening and closing inlet valves to adjust the point at which feed solution(s) are input and/or the point at which IVT reaction mixture is output, thereby adjusting the active length of the PFR and residence time of IVT reaction mixture. The flow rate of IVT reaction mixture through the PFR may also be modified to adjust its residence time. Modifications may be repeated until IVT reaction mixture residence time is within a desired tolerance (e.g., 80% to 120% of target residence time).

### Example 6: PFR System Alterations in Response to Raman Spectroscopy Results from the End of the PFR System

A Raman sensor is positioned at an output end of a PFR. Analysis of spectra collected by this Raman sensor determines whether the outlet stream from the PFR has reached a desired endpoint (e.g., NTP concentration below a given threshold). If the desired endpoint has not been reached by the time IVT reaction mixture reaches the output end, residence time may be increased. Residence time may be increased by moving the position(s) at which one or more feed solutions are input into the PFR farther upstream from the output end, thereby increasing the active length of the PFR. If IVT reaction mixture has reached the desired endpoint by the time it reaches the output end, residence time may be decreased (e.g., by moving the position of feed solution(s) introduction closer to the output end). Residence time may also be adjusted by modifying input feed rate(s) in addition or alternative to changing the position of feed input, such that the residence time of an IVT reaction mixture in the PFR is within a desired range (e.g., 80% to 120%) of the target residence time.

SEQUENCES
(T7 RNA polymerase)
>SEQ ID NO: 45
MNTINIAKNDESDIELAAIPENTLADHYGERLAREQLALEHESYE
MGEARFRKMFERQLKAGEVADNAAAKPLITTLLPKMIARINDWFE
EVKAKRGKRPTAFQFLQEIKPEAVAYITIKTTLACLTSADNTTVQ
AVASAIGRAIEDEARFGRIRDLEAKHFKKNVEEQLNKRVGHVYKK
AFMQVVEADMLSKGLLGGEAWSSWHKEDSIHVGVRCIEMLIESTG
MVSLHRQNAGVVGQDSETIELAPEYAEAIATRAGALAGISPMFQP
CVVPPKPWTGITGGGYWANGRRPLALVRTHSKKALMRYEDVYMPE
VYKAINIAQNTAWKINKKVLAVANVITKWKHCPVEDIPAIEREEL
PMKPEDIDMNPEALTAWKRAAAAVYRKDKARKSRRISLEFMLEQA
NKFANHKAIWFPYNMDWRGRVYAVSMFNPQGNDMTKGLLTLAKGK
PIGKEGYYWLKIHGANCAGVDKVPFPERIKFIEENHENIMACAKS
PLENTWWAEQDSPFCFLAFCFEYAGVQHHGLSYNCSLPLAFDGSC
SGIQHFSAMLRDEVGGRAVNLLPSETVQDIYGIVAKKVNEILQAD
AINGTDNEVVTVTDENTGEISEKVKLGTKALAGQWLAYGVTRSVT
KRSVMTLAYGSKEFGFRQQVLEDTIQPAIDSGKGLMFTQPNQAAG
YMAKLIWESVSVTVVAAVEAMNWLKSAAKLLAAEVKDKKTGEILR
KRCAVHWVTPDGFPVWQEYKKPIQTRLNLMFLGQFRLQPTINTNK
DSEIDAHKQESGIAPNFVHSQDGSHLRKTVVWAHEKYGIESFALI
HDSFGTIPADAANLFKAVRETMVDTYESCDVLADFYDQFADQLHE
SQLDKMPALPAKGNLNLRDILESDFAFA
(T7 RNA polymerase variant 1)
>SEQ ID NO: 46
MNTINIAKNDFSDIELAAIPENTLADHYGERLAREQLALEHESYE
MAEARFRKMFERQLKAGEVADNAAAKPLITTLLPKMIARINDWFE
EVKAKRGKRPTAFQFLQEIKPEAVAYITIKTTLACLTSADNTTVQ
AVASAIGRAIEDEARFGRIRDLEAKHFKKNVEEQLNKRVGHVYKK
AFMQVVEADMLSKGLLGGEAWSSWHKEDSIHVGVRCIEMLIESTG
MVSLHRQNAGVVGQDSETIELAPEYAEAIATRAGALAGISPMFQP
CVVPPKPWTGITGGGYWANGRRPLALVRTHSKKALMRYEDVYMPE
VYKAINIAQNTAWKINKKVLAVANVITKWKHCPVEDIPAIEREEL
PMKPEDIDMNPEALTAWKRAAAAVYRKDKARKSRRISLEFMLEQA
NKFANHKAIWFPYNMDWRGRVYAVSMFNPQGNDMTKGLLTLAKGK
PIGKEGYYWLKIHGANCAGVDKVPFPERIKFIEENHENIMACAKS
PLENTWWAEQDSPFCFLAFCFEYAGVQHHGLSYNCSLPLAFDGSC
SGIQHFSAMLRDEVGGRAVNLLPSETVQDIYGIVAKKVNEILQAD
AINGTDNEVVTVTDENTGEISEKVKLGTKALAGQWLAYGVTRSVT
KRSVMTLAYGSKEFGFRQQVLEDTIQPAIDSGKGLMFTQPNQAAG
YMAKLIWESVSVTVVAAVEAMNWLKSAAKLLAAEVKDKKTGEILR
KRCAVHWVTPDGFPVWQEYKKPIQTRLNLMFLGQFRLQPTINTNK
DSEIDAHKQESGIAPNFVHSQDGSHLRKTVVWAHEKYGIESFALI
HDSFGTIPADAANLFKAVRETMVDTYESCDVLADFYDQFADQLHE
SQLDKMPALPAKGNLNLRDILESDFAFAG
(T7 RNA polymerase variant 2)
>SEQ ID NO: 47
MNTINIAKNDFSDIELAAIPENTLADHYGERLAREQLALEHESYE
MAEARFRKMFERQLKAGEVADNAAAKPLITTLLPKMIARINDWFE
EVKAKRGKRPTAFQFLQEIKPEAVAYITIKTTLACLTSADNTTVQ
AVASAIGRAIEDEARFGRIRDLEAKHFKKNVEEQLNKRVGHVYKK
AFMQVVEADMLSKGLLGGEAWSSWHKEDSIHVGVRCIEMLIESTG
MVSLHRQNAGVVGQDSETIELAPEYAEAIATRAGALAGISPMFQP
CVVPPKPWTGITGGGYWANGRRPLALVRTHSKKALMRYEDVYMPE
VYKAINIAQNTAWKINKKVLAVANVITKWKHCPVWVIPAIEREEL
PMKPEDIDMNPEALTAWKRAAAAVYRKDKARKSRRISLEFMLEQA
NKFANHKAIWFPYNMDWRGRVYAVSMFNPQGNDMTKGLLTLAKGK
PIGKEGYYWLKIHGANCAGVDKVPFPERIKFIEENHENIMACAKS
PLENTWWAEQDSPFCFLAFCFEYAGVQHHGLSYNCSLPLAFDGSC
SGIQHFSAMLRDEVGGRAVNLLPSETVQDIYGIVAKKVNEILQAD
AINGTDNEVVTVTDENTGEISEKVKLGTKALAGQWLAYGVTRSVT
KRSVMTLAYGSKEFGFRQQVLEDTIQPAIDSGKGLMFTQPNQAAG
YMAKLIWESVSVTVVAAVEAMNWLKSAAKLLAAEVKDKKTGEILR
KRCAVHWVTPDGFPVWQEYKKPIQTRLNLMFLGQFRLQPTINTNK
DSEIDAHKQESGIAPNFVHSQDGSHLRKTVVWAHEKYGIESFALI
HDSFGTIPADAANLFKAVRETMVDTYESCDVLADFYDQFADQLHE
SQLDKMPALPAKGNLNLRDILESDFAFAG
(T7 RNA polymerase variant 3)
>SEQ ID NO: 48
MNTINIAKNDFSDIELAAIPENTLADHYGERLAREQLALEHESYE
MGEARFRKMFERQLKAGEVADNAAAKPLITTLLPKMIARINDWFE
EVKAKRGKRPTAFQFLQEIKPEAVAYITIKTTLACLTSADNTTVQ
AVASAIGRAIEDEARFGRIRDLEAKHFKKNVEEQLNKRVGHVYKK
AFMQVVEADMLSKGLLGGEAWSSWHKEDSIHVGVRCIEMLIESTG
MVSLHRQNAGVVGQDSETIELAPEYAEAIATRAGALAGISPMFQP
CVVPPKPWTGITGGGYWANGRRPLALVRTHSKKALMRYEDVYMPE
VYKAINIAQNTAWKINKKVLAVANVITKWKHCPVWVIPAIEREEL
PMKPEDIDMNPEALTAWKRAAAAVYRKDKARKSRRISLEFMLEQA
NKFANHKAIWFPYNMDWRGRVYAVSMFNPQGNDMTKGLLTLAKGK
PIGKEGYYWLKIHGANCAGVDKVPFPERIKFIEENHENIMACAKS
PLENTWWAEQDSPFCFLAFCFEYAGVQHHGLSYNCSLPLAFDGSC
SGIQHFSAMLRDEVGGRAVNLLPSETVQDIYGIVAKKVNEILQAD
AINGTDNEVVTVTDENTGEISEKVKLGTKALAGQWLAYGVTRSVT
KRSVMTLAYGSKEFGFRQQVLEDTIQPAIDSGKGLMFTQPNQAAG
YMAKLIWESVSVTVVAAVEAMNWLKSAAKLLAAEVKDKKTGEILR
KRCAVHWVTPDGFPVWQEYKKPIQTRLNLMFLGQFRLQPTINTNK
DSEIDAHKQESGIAPNFVHSQDGSHLRKTVVWAHEKYGIESFALI
HDSFGTIPADAANLFKAVRETMVDTYESCDVLADFYDQFADQLHE
SQLDKMPALPAKGNLNLRDILESDFAFA
(T7 RNA polymerase variant 4)
>SEQ ID NO: 49
MNTINIAKNDFSDIELAAIPENTLADHYGERLAREQLALEHESYE
MGEARFRKMFERQLKAGEVADNAAAKPLITTLLPKMIARINDWFE
EVKAKRGKRPTAFQFLQEIKPEAVAYITIKTTLACLTSADNTTVQ
AVASAIGRAIEDEARFGRIRDLEAKHFKKNVEEQLNKRVGHVYKK
AFMQVVEADMLSKGLLGGEAWSSWHKEDSIHVGVRCIEMLIESTG
MVSLHRQNAGVVGQDSETIELAPEYAEAIATRAGALAGISPMFQP
CVVPPKPWTGITGGGYWANGRRPLALVRTHSKKALMRYEDVYMPE
VYKAINIAQNTAWKINKKVLAVANVITKWKHCPVWVIPAIEREEL
PMKPEDIDMNPEALTAWKRAAAAVYRKDKARKSRRISLEFMLEQA
NKFANHKAIWFPYNMDWRGRVYAVSMFNPQGNDMTKGLLTLAKGK
PIGKEGYYWLKIHGANCAGVDKVPFPERIKFIEENHENIMACAKS
PLENTWWAEQDSPFCFLAFCFEYAGVQHHGLSYNCSLPLAFDGSC
SGIQHFSAMLRDEVGGRAVNLLPSETVQDIYGIVAKKVNEILQAD
AINGTDNEVVTVTDENTGEISEKVKLGTKALAGQWLAYGVTRSVT
KRSVMTLAYGSKEFGFRQQVLEDTIQPAIDSGKGLMFTQPNQAAG
YMAKLIWESVSVTVVAAVEAMNWLKSAAKLLAAEVKDKKTGEILR
KRCAVHWVTPDGFPVWQEYKKPIQTRLNLMFLGQFRLQPTINTNK
DSEIDAHKQESGIAPNFVHSQDGSHLRKTVVWAHEKYGIESFALI
HDSFGTIPADAANLFKAVRETMVDTYESCDVLADFYDQFADQLHE
SQLDKMPALPAKGNLNLRDILESDFAFA

It should be understood that any of the mRNA sequences may include a 5′ UTR and/or a 3′ UTR. The UTR sequences may be selected from the following sequences, or other known UTR sequences may be used. It should also be understood that any of the mRNA constructs may further comprise a poly(A) tail and/or cap (e.g., 7 mG(5′)ppp(5′) NlmpNp). Further, while many of the mRNAs and encoded antigen sequences include a signal peptide and/or a peptide tag (e.g., C-terminal His tag), it should be understood that the indicated signal peptide and/or peptide tag may be substituted for a different signal peptide and/or peptide tag, or the signal peptide and/or peptide tag may be omitted.

5′ UTR:
(SEQ ID NO: 1)
GGGAAAUAAGAGAGAAAAGAAGAGUAAGAAGAAAUAUAAGAGCC
ACC
5′ UTR:
(SEQ ID NO: 2)
GGGAAAUAAGAGAGAAAAGAAGAGUAAGAAGAAAUAUAAGACCCC
GGCGCCGCCACC
3′ UTR:
(SEQ ID NO: 3)
UGAUAAUAGGCUGGAGCCUCGGUGGCCAUGCUUCUUGCCCCUUGG
GCCUCCCCCCAGCCCCUCCUCCCCUUCCUGCACCCGUACCCCCGU
GGUCUUUGAAUAAAGUCUGAGUGGGCGGC
3′ UTR:
(SEQ ID NO: 4)
UGAUAAUAGGCUGGAGCCUCGGUGGCCUAGCUUCUUGCCCCUUG
GGCCUCCCCCCAGCCCCUCCUCCCCUUCCUGCACCCGUACCCCC
GUGGUCUUUGAAUAAAGUCUGAGUGGGCGGC

TABLE S-1
5′ UTR sequences

| SEQ ID | |
|---|---|
| NO: | Sequence |
| 5 | GGAAAUCGCAAAAUUUGCUCUUCGCGUUAGAUUUUUUUAGUUUUCUCGCAACUAGCAAG |
| | CUUUUUGUUCUCGCC |
| 6 | GGAAAUCCCCACAACCGCCUCAUAUCCAGGCUCAAGAAUAGAGCUCAGUGUUUUGUUGUU |
| | UAAUCAUUCCGACGUGUUUUGCGAUAUUCGCGCAAAGCAGCCAGUCGCGCGCUUGCUUUU |
| | AAGUAGAGUUGUUUUUCCACCCGUUUGCCAGGCAUCUUUAAUUUAACAUAUUUUUAUUUU |
| | UCAGGCUAACCUACGCCGCCACC |
| 7 | GGAAAUAAGAGAGAAAAGAAGAGUAAGAAGAAAUAUAAGAUCUCCCUGAGCUUCAGGGAG |
| | CCCCGGCGCCGCCACC |
| 8 | GGAAACCCCCCACCCCCGUAAGAGAGAAAAGAAGAGUAAGAAGAAAUAUAAGAUCUCCCU |
| | GAGCUUCAGGGAGCCCCGGCGCCGCCACC |
| 9 | GGAGAACUUCCGCUUCCGUUGGCGCAAGCGCUUUCAUUUUUUCUGCUACCGUGACUAAG |
| 10 | GGAAAUAAGAGAGAAAAGAAGAGUAAGAAGAAAUAUAAGAGCCACC |
| 11 | GGAAAUAAGAGAGAAAAGAAGAGUAAGAAGAAAUAUAAGACCCCGGCGCCGCCACC |
| 12 | GGAAAUCGCAAAAUUUGCUCUUCGCGUUAGAUUUCUUUUAGUUUUCUCGCAACUAGCAAG |
| | CUUUUUGUUCUCGCCGCCGCC |
| 13 | GGAAAUCGCAAAAUUUUCUUUUCGCGUUAGAUUUCUUUUAGUUUUCUUUCAACUAGCAAG |
| | CUUUUUGUUCUCGCCGCCGCC |
| 14 | G G A A A U C G C A A A A (N₂)ₓ (N₃)ₓ C U (N₄)ₓ (N₅)ₓ C G C G U |
| | U A G A U U U C U U U U A G U U U U C U N₆ N₇ C A A C U A G C |
| | A A G C U U U U U G U U C U C G C C (N₈ C C)ₓ |
| | (N₂)ₓ is a uracil and x is an integer from 0 to 5, |
| | e.g., wherein x = 3 or 4; |
| | (N₃)ₓ is a guanine and x is an integer from 0 to 1; |
| | (N₄)ₓ is a cytosine and x is an integer from 0 to 1; |
| | (N₅)ₓ is a uracil and x is an integer from 0 to 5, |
| | e.g., wherein x = 2 or 3; |
| | N₆ is a uracil or cytosine; |
| | N₇ is a uracil or guanine; |
| | N₈ is adenine or guanine and x is an integer from 0 to 1. |
| 15 | GGAAAAUUUUAGCCUGGAACGUUAGAUAACUGUCCUGUUGUCUUUAUAUACUUGGUCCCC |
| | AAGUAGUUUGUCUUCCAAA |
| 16 | GGAAACUUUAUUUAGUGUUACUUUAUUUUCUGUUUAUUUGUGUUUCUUCAGUGGGUUUGU |
| | UCUAAUUUCCUUGGCCGCC |
| 17 | GGAAAAUCUGUAUUAGGUUGGCGUGUUCUUUGGUCGGUUGUUAGUAUUGUUGUUGAUUCG |
| | UUUGUGGUCGGUUGCCGCC |
| 18 | GGAAAAUUAUUAACAUCUUGGUAUUCUCGAUAACCAUUCGUUGGAUUUUAUUGUAUUCGU |
| | AGUUUGGGUUCCUGCCGCC |
| 19 | GGAAAUUAUUAUUAUUUCUAGCUACAAUUUAUCAUUGUAUUAUUUUAGCUAUUCAUCAUU |
| | AUUUACUUGGUGAUCAACA |
| 20 | GGAAAUAGGUUGUUAACCAAGUUCAAGCCUAAUAAGCUUGGAUUCUGGUGACUUGCUUCA |
| | CCGUUGGCGGGCACCGAUC |
| 21 | GGAAAUCGUAGAGAGUCGUACUUAGUACAUAUCGACUAUCGGUGGACACCAUCAAGAUUA |
| | UAAACCAGGCCAGA |
| 22 | GGAAACCCGCCCAAGCGACCCCAACAUAUCAGCAGUUGCCCAAUCCCAACUCCCAACACA |
| | AUCCCCAAGCAACGCCGCC |
| 23 | GGAAAGCGAUUGAAGGCGUCUUUUCAACUACUCGAUUAAGGUUGGGUAUCGUCGUGGGAC |
| | UUGGAAAUUUGUUGUUUCC |
| 24 | GGAAACUAAUCGAAAUAAAAGAGCCCCGUACUCUUUUAUUUCUAUUAGGUUAGGAGCCUU |
| | AGCAUUUGUAUCUUAGGUA |
| 25 | GGAAAUGUGAUUUCCAGCAACUUCUUUUGAAUAUAUUGAAUUCCUAAUUCAAAGCGAACA |
| | AAUCUACAAGCCAUAUACC |
| 26 | GGAAAUCGUAGAGAGUCGUACUUACGUGGUCGCCAUUGCAUAGCGCGCGAAAGCAACAGG |
| | AACAAGAACGCGCC |
| 27 | GGAAAUCGUAGAGAGUCGUACUUAGAAUAAACAGAGUCGGGUCGACUUGUCUCUGAUACU |
| | ACGACGUCACAAUC |
| 28 | GGAAAAUUUGCCUUCGGAGUUGCGUAUCCUGAACUGCCCAGCCUCCUGAUAUACAACUGU |
| | UCCGCUUAUUCGGGCCGCC |
| 29 | GGAAAUCUGAGCAGGAAUCCUUUGUGCAUUGAAGACUUUAGAUUCCUCUCUGCGGUAGAC |
| | GUGCACUUAUAAGUAUUUG |
| 30 | GGAAAGCGAUUGAAGGCGUCUUUUCAACUACUCGAUUAAGGUUGGGUAUCGUCGUGGGAC |
| | UUGGAAAUUUGUUGCCACC |
| 31 | GGAAAUUUUUUUUUGAUAUUAUAAGAGUUUUUUUUUGAUAUUAAGAAAAUUUUUUUUUGA |
| | UAUUAGAAGAGUAAGAAGAAAUAUAAGACCCCGGCGCCGCCACC |
| 32 | GGAAAUAAGAGAGAAAAGAAGAGUAAGAAGAAAUAUAAGAGCCAAAAAAAAAAAACC |
| 33 | GGAAAUCUCCCUGAGCUUCAGGGAGUAAGAGAGAAAAGAAGAGUAAGAAGAAAUAUAAGA |
| | CCCCGGCGCCGCCACC |
| 34 | GCCRCC, wherein R = A or G |
| 35 | GGACUCACUAUUUGUUUUCGCGCCCAGUUGCAAAAA |

TABLE S-2
3′ UTR sequences (stop cassette is italicized;
miR binding sites are boldened)

| SEQ ID | |
|---|---|
| NO: | Sequence |
| 36 | UAAAGCUCCCCGGGGGCCUCGGUGGCCUAGCUUCUUGCCCCUUGGGCCUCCCCCCAGCC |
| | CCUCCUCCCCUUCCUGCACCCGUACCCCCGUGGUCUUUGAAUAAAGUCUGAGUGGGCGG |
| | C |
| 37 | UAAGUCUAAGCUGGAGCCUCCUGAGAGACCUGUGUGAACUAUUGAGAAGAUCGGAACAG |
| | CUCCUUACUCUGAGGAAGUUGGUACCCCCGUGGUCUUUGAAUAAAGUCUGAGUGGGCGG |
| | C |
| 38 | UAAAGCUCCCCGGGGCAAACACCAUUGUCACACUCCAGCCUCGGUGGCCUAGCUUCUUG |
| | CCCCUUGGGCCCAAACACCAUUGUCACACUCCAUCCCCCCAGCCCCUCCUCCCCUUCCU |
| | GCACCCGUACCCCCCAAACACCAUUGUCACACUCCAGUGGUCUUUGAAUAAAGUCUGAG |
| | UGGGCGGC |
| | (miR122 binding sites boldened) |
| 39 | UAAAGCUCCCCGGGGUCCAUAAAGUAGGAAACACUACAGCUGGAGCCUCGGUGGCCUAG |
| | CUUCUUGCCCCUUGGGCCCAAACACCAUUGUCACACUCCAUCCCCCCAGCCCCUCCUCC |
| | CCUUCCUGCACCCGUACCCCCGUGGUCUUUGAAUAAAGUCUGAGUGGGCGGC |
| | (miR-142-3p and miR122 binding sites boldened) |
| 40 | UAAAGCUCCCCGGGGGCCUCGGUGGCCUAGCUUCUUGCCCCUUGGGCCUCCCCCCAGCC |
| | CCUCCUCCCCUUCCUGCACCCGUACCCCCCAAACACCAUUGUCACACUCCAGUGGUCUU |
| | UGAAUAAAGUCUGAGUGGGCGGC |
| | (miR122 binding site boldened) |
| 41 | UAAGCCCCUCCGGGGCAAACACCAUUGUCACACUCCAGCCUCGGUGGCCUAGCUUCUUGC |
| | CCCUUGGGCCCAAACACCAUUGUCACACUCCAUCCCCCCAGCCCCUCCUCCCCUUCCUGC |
| | ACCCGUACCCCCCAAACACCAUUGUCACACUCCAGUGGUCUUUGAAUAAAGUCUGAGUGG |
| | GGGGC |
| | (miR122 binding sites boldened) |
| 42 | UAAGCCCCUCCGGGGUCCAUAAAGUAGGAAACACUACAGCCUCGGUGGCCUAGCUUCUUG |
| | CCCCUUGGGCCUCCAUAAAGUAGGAAACACUACAUCCCCCCAGCCCCUCCUCCCCUUCCU |
| | GCACCCGUACCCCCCGCAUUAUUACUCACGGUACGAGUGGUCUUUGAAUAAAGUCUGAGU |
| | GGGCGGC |
| | (miR-142-3p and miR-126-3p binding sites boldened) |
| 43 | UAAGCCCCUCCGGGGUCCAUAAAGUAGGAAACACUACAGCUGGAGCCUCGGUGGCCUAG |
| | CUUCUUGCCCCUUGGGCCCAAACACCAUUGUCACACUCCAUCCCCCCAGCCCCUCCUCC |
| | CCUUCCUGCACCCGUACCCCCGUGGUCUUUGAAUAAAGUCUGAGUGGGCGGC |
| | (miR-142-3p and miR122 binding sites boldened) |
| 44 | UAAGCCCCUCCGGGGGCCUCGGUGGCCUAGCUUCUUGCCCCUUGGGCCUCCCCCCAGCC |
| | CCUCCUCCCCUUCCUGCACCCGUACCCCCCAAACACCAUUGUCACACUCCAGUGGUCUU |
| | UGAAUAAAGUCUGAGUGGGCGGC |
| | (miR122 binding site boldened) |

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between and including the upper and lower ends of the range are specifically contemplated and described herein.

1. An in vitro transcription (IVT) method, the method comprising:
(i) in a continuous reaction apparatus, incubating an IVT reaction mixture comprising a buffer, magnesium, a DNA, an RNA polymerase, a cap analog, adenosine triphosphate (ATP), cytidine triphosphate (CTP), guanosine triphosphate (GTP), and uridine triphosphate (UTP), whereby the RNA polymerase transcribes the DNA to produce an mRNA;
wherein the IVT reaction mixture is formed by adding a first feed solution to the continuous reaction apparatus at a first input feed rate and a second feed solution to the continuous reaction apparatus at a second input feed rate,
wherein the IVT reaction mixture is output from the continuous reaction apparatus at a first output flow rate;
(iii) obtaining Raman spectra from the IVT reaction mixture over time;
(iv) determining a reaction rate and a target endpoint from the Raman spectra;
(v) determining a target residence time from the reaction rate and target endpoint;
(vi) modifying the first input feed rate, second input feed rate, and/or first output flow rate such that a residence time of the IVT reaction mixture in the continuous reaction apparatus is 80% to 120% of the target residence time.

2. The method of claim 1, wherein the target residence time is determined by calculating a target reaction volume from the Raman spectra.

3. The method of claim 1, wherein the residence time of the IVT reaction mixture is modified by modifying a total input feed rate, the total input feed rate being the sum of the first input feed rate and the second input feed rate.

4. The method of claim 1, wherein the residence time of the IVT reaction mixture is modified by modifying the first output flow rate.

5. An in vitro transcription method, the method comprising:
(i)(a) in a preliminary reaction apparatus, incubating a preliminary in vitro transcription (IVT) reaction mixture comprising a buffer, magnesium, a DNA, an RNA polymerase, a cap analog, adenosine triphosphate (ATP), cytidine triphosphate (CTP), guanosine triphosphate (GTP), and uridine triphosphate (UTP), whereby the RNA polymerase transcribes the DNA to produce an mRNA;
(i)(b) obtaining Raman spectra from the preliminary IVT reaction mixture over time;
(i)(c) determining a reaction rate and a target endpoint from the Raman spectra;
(i)(d) determining a target residence time from the reaction rate and target endpoint; and
(ii) in a continuous reaction apparatus comprising a plug flow reactor (PFR), incubating an in vitro transcription (IVT) reaction mixture flowing through the PFR at 80% to 120% of the target residence time, the IVT reaction mixture comprising a buffer, magnesium, a DNA, an RNA polymerase, a cap analog, adenosine triphosphate (ATP), cytidine triphosphate (CTP), guanosine triphosphate (GTP), and uridine triphosphate (UTP), whereby the RNA polymerase transcribes the DNA to produce the mRNA,
wherein the IVT reaction mixture is output from the continuous reaction apparatus at a first output flow rate.

6. An in vitro transcription (IVT) method, the method comprising, in a continuous reaction apparatus comprising a plug flow reactor (PFR):
(i) incubating an in vitro transcription (IVT) reaction mixture flowing through the PFR with a residence time, the IVT reaction mixture comprising a buffer, magnesium, a DNA, an RNA polymerase, a cap analog, adenosine triphosphate (ATP), cytidine triphosphate (CTP), guanosine triphosphate (GTP), and uridine triphosphate (UTP);
(ii) obtaining Raman spectra from the IVT reaction mixture (a) at two or more points along the PFR separated by a predetermined distance, or (b) outlet location of the PFR, over time;
(iii) determining a reaction rate and a target endpoint from the Raman spectra;
(iv) determining a target residence time from the reaction rate and target endpoint; and
(v) modifying the residence time such that the IVT reaction mixture flows through the PFR with 80% to 120% of the target residence time,
wherein the IVT reaction mixture is output from the continuous reaction apparatus at a first output flow rate.

7. The method of claim 6, wherein step (ii) comprises obtaining Raman spectra from the IVT reaction mixture at the outlet location of the PFR over time.

8. (canceled)

9. The method of claim 6, wherein an active length of the PFR is adjustable, wherein modifying the residence time comprises opening a valve upstream of a current outlet location, after determining that the target endpoint occurred prior to the IVT reaction mixture reaching an end of the active length of the PFR.

10. The method of claim 9, wherein modifying the residence time comprises closing an outlet and opening a valve downstream of the outlet, after determining that the target endpoint did not occur prior to the IVT reaction mixture reaching an end of the active length of the PFR.

11. The method of claim 1, wherein an mRNA yield of at least 80% of a theoretical maximum mRNA yield occurs when the residence time of the IVT reaction mixture in the continuous reaction apparatus is the target residence time.

12. The method of claim 1, wherein a reaction rate of at least 80% of a theoretical maximum reaction rate occurs when the residence time of the IVT reaction mixture in the continuous reaction apparatus is the target residence time.

13. The method of claim 1, wherein a concentration of nucleotide triphosphates (NTPs) in the IVT reaction mixture being output at the first output flow rate is 20% or less of a concentration of NTPs input into the continuous reaction apparatus.

14. The method of claim 1, wherein the IVT reaction mixture output at the first output flow rate flows into an additional reaction apparatus, and wherein the method further comprises:
(i) contacting the additional reaction apparatus with an additional feed solution comprising an additional buffer and a DNase to form a DNase reaction mixture; and
(ii) incubating the DNase reaction mixture, whereby the DNase cleaves the DNA to produce one or more DNA fragments; and
(iii) separating the mRNA from the one or more DNA fragments and one or more other impurities to obtain an isolated mRNA composition.

15. The method of claim 14, wherein the IVT reaction mixture flowing at the first output flow rate flows continuously into the additional reaction apparatus.

16. (canceled)

17. The method of claim 14, wherein the additional reaction apparatus is a continuous plug flow reactor (CPFR) having one or more curved pipes, wherein the DNase reaction mixture flows through the CPFR with a Dean number (De) of at least 30, wherein each of the one or more curved pipes comprises (a) a diameter, and (b) a curve having a radius that is 180% to 400% of the diameter, wherein the additional reaction apparatus has a pressure drop of 0.5 bar or less.

18.-30. (canceled)

31. An apparatus comprising:
(i) a first feed solution container;
(ii) a second feed solution container;
(iii)(a) a continuous in vitro transcription (IVT) reaction apparatus fluidically coupled downstream of both the first feed solution container and the second feed solution container which is configured to receive a first mixed inlet stream; and
(iii)(b) a Raman sensor coupled to the continuous IVT reaction apparatus.

32. The apparatus of claim 31, wherein the continuous IVT reaction apparatus is a plug flow reaction (PFR) comprising two or more Raman sensors configured to obtain Raman spectra from a solution flowing through the PFR at two or more points separated by a predetermined distance.

33. The apparatus of claim 32, wherein the continuous IVT reaction apparatus is a plug flow reaction (PFR), wherein the Raman sensor is configured to obtain a Raman spectrum from an output end of the continuous IVT reaction apparatus.

34. (canceled)

35. The apparatus of claim 31, further comprising a DNase reaction apparatus (a) fluidically coupled downstream of the continuous IVT reaction apparatus, and (b) configured to receive a third feed solution comprising a DNase, the DNase reaction apparatus comprising a continuous plug flow reaction (CPFR) comprising one or more curved pipes, wherein each of the one or more curved pipes comprises (a) a diameter, and (b) a curve having a radius that is 180% to 400% of the diameter, wherein the DNase reaction apparatus has a pressure drop of 0.5 bar or less.

36.-37. (canceled)

38. The apparatus of claim 35, further comprising:
(a) a tangential flow filtration module; and
(b) an oligo-dT chromatography module,
wherein the apparatus is configured to remove one or more DNA fragments from a mixture comprising an mRNA and the one or more DNA fragments, before the mRNA is introduced into the oligo-dT chromatography module.
